(19) United States
(12) Patent Application Publication
(10) Pub. No.: US 2004/0110287 A1
(43) Pub. Date: Jun. 10, 2004

(54) Multi-step method for the differentiation of insulin positive, glucose responsive cells

(75) Inventors: Diana Clarke, Cambridge, MA (US); Josephine S. D'Alessandro, Marblehead, MA (US); Kuanghui Lu, Brookline, MA (US); Anlai Wang, Newton, MA (US)

Correspondence Address: ROPES & GRAY LLP, ONE INTERNATIONAL PLACE, BOSTON, MA, 02110-2624, US

(73) Assignee: ES Cell International Pte Ltd.

(21) Appl. No.: 10/629,933

(22) Filed: Jul. 29, 2003

Related U.S. Application Data
(60) Provisional application No. 60/399,476, filed on Jul. 29, 2002.
(60) Provisional application No. 60/409,847, filed on Sep. 11, 2002.
(60) Provisional application No. 60/452,732, filed on Mar. 7, 2003.

(51) Int. Cl. C12N 5/08

(52) U.S. Cl. 435/366

(57) ABSTRACT

The present invention provides improved methods of differentiating insulin+, glucose responsive islet-like structures from insulin− cells. The invention further provides methods for using insulin+, glucose responsive islet-like structures, as well as the insulin+, glucose responsive cells which comprise said islet-like clusters.

### RELATED APPLICATIONS

This application claims priority to U.S. provisional application No. 60/399,476 filed Jul. 29, 2002, U.S. provisional application No. 60/409,847 filed Sep. 11, 2002, and U.S. provisional application No. 60/452,732 filed Mar. 7, 2003, the disclosures of which are hereby incorporated by reference in their entirety.

### BACKGROUND OF THE INVENTION

Pluripotent stem cells have generated tremendous interest in the biomedical community. With the realization that stem cells can be isolated from many adult, fetal, and embryonic tissues has come the hope that cultures of relatively pure stem cells can be maintained in vitro for use in treating a wide range of conditions. Stem cells, with their capability for self-regeneration in vitro and their ability to produce differentiated cell types, may be useful for replacing the function of aging or failing cells in nearly any organ system. By some estimates, over 100 million Americans suffer from disorders that might be alleviated by transplantation technologies that utilize stem cells (Perry (2000) Science 287: 1423). Such illnesses include, for example, cardiovascular diseases, autoimmune diseases, diabetes, osteoporosis, cancers and burns.

Insulin-dependent diabetes mellitus (IDDM) is a good example of a disease that could be cured or ameliorated through the use of stem cells. Insulin-dependent diabetes mellitus is a disease characterized by elevated blood glucose and the absence of the hormone insulin. The cause of the raised glucose levels is insufficient secretion of the hormone insulin by the pancreas. In the absence of this hormone, the body's cells are not able to absorb glucose from the blood stream causing an accumulation in the blood. Chronically elevated blood glucose damages tissues and organs. IDDM is treated with insulin injections. The size and timing of insulin injections are influenced by measurements of blood glucose.

There are over 400 million diabetics in the world today. Diabetes is one of the most prevalent chronic diseases in the United States, and a leading cause of death. Estimates based on the 1993 National Health Interview Survey (NHIS) indicate that diabetes has been diagnosed in 1% of the U.S. population age <45 years, 6.2% of those age 45-64 years, and 10.4% of those age >65 years. In other terms, in 1993 an estimated 7.8 million persons in the United States were reported to have this chronic condition. In addition, based on the annual incidence rates for diabetes, it is estimated that about 625,000 new cases of diabetes are diagnosed each year, including 595,000 cases of non-insulin-dependent diabetes mellitus (NIDDM) and 30,000 cases of insulin-dependent diabetes mellitus (IDDM). Persons with diabetes are at risk for major complications, including diabetic ketoacidosis, end-stage renal disease, diabetic retinopathy and amputation. There are also a host of less directly related conditions, such as hypertension, heart disease, peripheral vascular disease and infections, for which persons with diabetes are at substantially increased risk.

While medications such as injectable insulin and oral hypoglycemics allow diabetics to live longer, diabetes remains the third major killer, after heart disease and cancer. Diabetes is also a very disabling disease, because medications do not control blood glucose levels well enough to prevent swinging between high and low blood glucose levels, with resulting damage to the kidneys, eyes, and blood vessels.

Replenishment of functional glucose-sensing, insulin-secreting pancreatic beta cells through islet transplantation has been a longstanding therapeutic target. The limiting factor in this approach is the availability of an islet source that is safe, reproducible, and abundant. Current methodologies use either cadaverous material or porcine islets as transplant substrates (Korbutt et al. (1997) Adv. Exp. Med. Biol. 426: 397-410). However, significant problems to overcome are the low availability of donor tissue, the variability and low yield of islets obtained via dissociation, and the enzymatic and physical damage that may occur as a result of the isolation process (reviewed by Secchi et al. (1997) Horm. Metab. Res. 29: 1-8; Sutherland et al. (1996) Transplant Proc. 28: 2131-2133). In addition are issues of immune rejection and current concerns with xenotransplantation using porcine islets (reviewed by Weir & Bonner-Weir (1997) 46: 1247-1256).

### SUMMARY OF THE INVENTION

Diabetes is a serious disorder that exacts a tremendous toll both financially, and in terms of its impact on the quality of life of its sufferers. One attractive potential treatment for diabetes, as well as for other conditions including injuries and diseases of the pancreas and diseases which affect the body's ability to properly respond to glucose, involves the use of stem cells to replace lost or damaged cell types. In the case of diabetes, damaged β-cells could be replaced either via transplantation of stem cells which would differentiate in vivo, by the transplantation of β-cells differentiated ex vivo, or by the transplantation of differentiated islets containing β-cells. Additionally, although much of the focus has been on the differentiation of β-cells from stem cells, any cell type (stem or committed) which can be influenced to differentiate to give rise to glucose responsive, β-cells would be useful for the treatment of diabetes or other conditions which result in the damage or destruction of functional β-cells.

Despite the great therapeutic potential of stem cells, and their differentiated progeny, there are several serious limitations which have prevented the widespread realization of stem cell treatments. Adult stem cells are quite rare, and previous methods to culture and differentiate stem cells along particular lineages have yielded promising but very inefficient results. In order for therapeutic methods employing stem cells to become a reasonable treatment option for a variety of diseases such as diabetes, there exists a need for improved methods for purifying stem cells and differentiating such stem cells along particular lineages. Furthermore, there is a need for improved methods of expanding, in a given tissue sample, the number of cells capable of differentiating along a particular lineage.

In addition to a need for more efficient methods for differentiating stem cells, there also exists a need for improved methods of differentiating mature cell types (either from stem cells or from more committed cell populations) capable of functioning as the endogenous cell types function. For example, although methods may exist to influence the differentiation of a cell to express a marker of neuronal differentiation, that cell must ultimately be able to function as a neuron (i.e., to transmit/respond to neurotransmitters). Therapeutic intervention for diabetes requires not only cells which express markers of pancreatic differentiation (i.e., insulin) but also cells which are glucose responsive. The present invention provides improved methods for differentiating cells which not only express markers of pancreatic endocrine differentiation, but are also responsive to glucose (e.g., for example by secreting insulin in response to elevated plasma glucose levels). Such cells provide the basis for improved methods of treating injuries and disorders of the pancreas, as well as other disorders which affect the body's ability to properly respond to glucose.

The present invention provides improved methods for differentiating insulin+, glucose responsive cells. The invention contemplates that such insulin+, glucose responsive cells may be differentiated from stem cells (including adult stem cells, fetal stem cells, and embryonic stem cells), as well as from more committed tissue. The present invention further provides the isolated islet-like structures differentiated using the disclosed methods. These islet-like structures contain insulin+, glucose responsive cells, as well as somatostatin+ and glucagon+ cells. The invention further provides methods for treating patients by transplanting a therapeutically effective amount of the islet-like structures of the invention.

In one aspect, the invention provides a method for culturing substantially purified, insulin− cells, wherein said cells differentiate to insulin+, glucose responsive cells.

In one embodiment, the insulin− cells are stem cells.

In one embodiment, the insulin− cells are cytokeratin+.

In one embodiment, the insulin− cells are cytokeratin−.

In one embodiment, the substantially purified population of cells is at least about 50%, but more preferably about 60%, 70%, 80% or most preferably about 90%, 95%, or 99% pure. In another embodiment, the purified population of cells has fewer than about 20%, more preferably fewer than about 10%, most preferably fewer than about 5% of lineage committed cells. In the context of the present invention, a lineage committed cell is one that expresses one or more of the following markers of a differentiated endocrine cell: insulin, somatostatin, or glucagon.

In one embodiment, the insulin+ cells are also pdx1+.

In one embodiment, the insulin− cells are isolated from pancreatic tissue.

In another embodiment, the insulin− cells are isolated from duct or tubule tissue. In another embodiment, the duct or tubule tissue is selected from the group consisting of pancreatic duct, hepatic duct, kidney duct, kidney tubule (e.g., proximal tubule, distal tubule), bile duct, tear duct, lactiferous duct, ejaculatory duct, seminiferous tubule, efferent duct, cystic duct, lymphatic duct, and thoracic duct.

In another embodiment, the insulin− cells are stem cells selected from the group consisting of embryonic stem cells, fetal stem cells, and adult stem cells. In one embodiment, the adult stem cells are selected from the group consisting of neural stem cells, neural crest stem cells, pancreatic stem cells, skin-derived stem cells, cardiac stem cells, liver stem cells, endothelial stem cells, hematopoietic stem cells, and mesenchymal stem cells. In another embodiment, the adult stem cells are isolated from an adult tissue. In yet another embodiment, the stem cells are isolated from an adult tissue selected from the group consisting of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, and olfactory epithelium.

In another embodiment, the insulin− cells differentiate to form islet-like structures containing insulin+ cells. In a preferred embodiment, the insulin+ cells are glucose responsive. In another preferred embodiment, the islet-like structures additionally contain glucagon+ and somatostatin+ cells. In still another preferred embodiment, the glucagon+ and somatostatin+ cells are localized to the periphery of the islet-like structure.

In a second aspect, the invention provides a method for differentiating substantially purified, insulin− cells to insulin+, glucose responsive cells. The method comprises the following steps: (a) culturing substantially purified cells as non-adherent spheres; (b) selecting cells by culturing in the presence of a gp130 agonist; (c) dissociating the spheres and culturing in the presence of mitogens, wherein at least one mitogen is an FGF family member; (d) culturing the spheres in the presence of at least two growth factors, or growth factor agonists, wherein at least one growth factor is an FGF family member; (e) plating the spheres on a coated substratum in high-glucose media; and (f) culturing the spheres in media containing standard glucose.

In one embodiment, the insulin− cells are stem cells.

In one embodiment, the insulin− cells are cytokeratin+.

In one embodiment, the insulin− cells are cytokeratin−.

In one embodiment, the gp130 agonist recited in step (b) is selected from the group consisting of cardiotrophin-1, LIF, oncostatin M, IL-6, IL-11, ciliary neurotrophic factor, and granulocyte colony stimulating factor.

In another embodiment, the FGF family member recited in step (c) or (d) is independently selected from the group consisting of FGF-5, FGF-7, FGF-8, FGF-10, FGF-16, FGF-17, and FGF-18. In a preferred embodiment, the FGF family member recited in step (c) or (d) is independently selected from the group consisting of FGF-8, FGF-17, and FGF-18.

In another embodiment, step (c) includes a hedgehog polypeptide selected from the group consisting of sonic hedgehog, Indian hedgehog, and desert hedgehog. The polypeptide may be a full length polypeptide, or an active fragment which can activate hedgehog signaling. Furthermore, the hedgehog polypeptide, or active fragment thereof, may be modified with one or more lipophilic or other moieties that increase the hydrophobicity of the polypeptide. In another embodiment, step (c) includes a hedgehog agonist selected from the group consisting of a hedgehog polypeptide or a small molecule which can potentiate hedgehog signaling.

In any of the foregoing embodiments, step (c) and/or (d) may include heparin.

In another embodiment, the growth factors of step (d) are family members selected from the group consisting of EGF, FGF, IGF-1, IGF-11, TGF-α, TGF-β, PDGF, VEGF, and hedgehog.

In another embodiment, the coated substratum of step (e) comprises at least one of poly-L-ornithine, laminin, fibronectin, or superfibronectin. In a preferred embodiment, the coated substratum comprises superfibronectin.

In another embodiment, the coated substratum of step (e) comprises Matrigel or a cellular feeder layer.

In another embodiment, the high-glucose media of step (e) comprises at least 10 mM glucose. In another embodiment, the high-glucose media of step (e) comprises at least 11 mM glucose. The glucose in the medium can range from 10-17 mM in step (e).

In another embodiment, step (e) includes media containing at least one factor selected from the group consisting of serum, PYY, HGF, and forskolin.

In another embodiment, step (e) includes at least one cAMP elevating agent. In a preferred embodiment, the cAMP elevating agent is selected from the group consisting of CPT-cAMP, forskolin, Na-Butyrate, isobutyl methylxanthine, cholera toxin, 8-bromo-cAMP, dibutyryl-cAMP, dioctanoyl-cAMP, pertussis toxin, prostaglandins, colforsin, β-adrenergic receptor agonists, and cAMP analogs. In another preferred embodiment, the cAMP elevating agent is forskolin. In another embodiment, at least one cAMP elevating agent is an inhibitor of cAMP phosphodiesterase.

In another embodiment, the standard glucose media of step (f) comprises less than 7.5 mM glucose. In another embodiment, the standard glucose media of step (f) comprises less than 6 mM glucose. In still another embodiment, the standard glucose media of step (f) comprises less than 5.5 mM glucose.

In another embodiment, the media of step (f) additionally comprises at least one factor selected from the group consisting of serum, leptin, nicotinamide, malonyl CoA, and exendin-4.

In one embodiment, the insulin− cells are isolated from pancreatic tissue.

In another embodiment, the insulin− cells are isolated from duct or tubule tissue. In another embodiment, the duct or tubule tissue is selected from the group consisting of pancreatic duct, hepatic duct, kidney duct, kidney tubule (e.g., proximal tubule, distal tubule), bile duct, tear duct, lactiferous duct, ejaculatory duct, seminiferous tubule, efferent duct, cystic duct, lymphatic duct, and thoracic duct.

In another embodiment, the insulin− cells are stem cells selected from the group consisting of embryonic stem cells, fetal stem cells, and adult stem cells. In one embodiment, the adult stem cells are selected from the group consisting of neural stem cells, neural crest stem cells, pancreatic stem cells, skin-derived stem cells, cardiac stem cells, liver stem cells, endothelial stem cells, hematopoietic stem cells, and mesenchymal stem cells. In another embodiment, the adult stem cells are isolated from an adult tissue. In yet another embodiment, the stem cells are isolated from an adult tissue selected from the group consisting of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, and olfactory epithelium.

In another embodiment, the insulin− cells differentiate to form islet-like structures containing insulin+ cells. In a preferred embodiment, the islet-like structures additionally contain glucagon+ and somatostatin+ cells. In another preferred embodiment, the glucagon+ and somatostatin+ cells are localized to the periphery of the islet-like structure.

In a third aspect, the invention provides a method for differentiating substantially purified, insulin− cells to insulin+, glucose responsive cells. The method comprises the following steps: (a) culturing substantially purified cells as non-adherent spheres; (b) selecting cells by culturing in serum-free media supplemented with cardiotrophin-1; (c) dissociating the spheres and culturing in serum-free media supplemented with FGF-18 and a hedgehog polypeptide; (d) culturing the spheres in the presence of at least two growth factors, or growth factor agonists, wherein at least one growth factor is FGF-18; (e) plating the spheres on a coated substratum in high-glucose media; and (f) culturing the spheres in media containing standard glucose supplemented with nicotinamide.

In one embodiment, the insulin− cells are stem cells.

In one embodiment, the insulin− cells are cytokeratin+.

In one embodiment, the insulin− cells are cytokeratin−.

In one embodiment, the media of step (c) includes heparin.

In another embodiment, the growth factors of step (d) are members of a growth factor family selected from the group consisting of EGF, FGF, TGF-α, TGF-β, IGF-1, IGF-11, PDGF, VEGF, and hedgehog. In another embodiment, the media of step (d) optionally includes heparin.

In another embodiment, the coated substratum of step (e) comprises at least one of poly-L-ornithine, laminin, fibronectin, or superfibronectin. In a preferred embodiment, the coated substratum of step (e) comprises superfibronectin.

In another embodiment, the coated substratum of step (e) comprises Matrigel or a cellular feeder layer.

In one embodiment, the insulin− cells are isolated from pancreatic tissue.

In another embodiment, the insulin− cells are isolated from duct or tubule tissue. In another embodiment, the duct or tubule tissue is selected from the group consisting of pancreatic duct, hepatic duct, kidney duct, kidney tubule (e.g., proximal tubule, distal tubule), bile duct, tear duct, lactiferous duct, ejaculatory duct, seminiferous tubule, efferent duct, cystic duct, lymphatic duct, and thoracic duct.

In another embodiment, the insulin− cells are stem cells selected from the group consisting of embryonic stem cells, fetal stem cells, and adult stem cells. In one embodiment, the adult stem cells are selected from the group consisting of neural stem cells, neural crest stem cells, pancreatic stem cells, skin-derived stem cells, cardiac stem cells, liver stem cells, endothelial stem cells, hematopoietic stem cells, and mesenchymal stem cells. In another embodiment, the adult stem cells are isolated from an adult tissue. In yet another embodiment, the stem cells are isolated from an adult tissue selected from the group consisting of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, and olfactory epithelium.

In a fourth aspect, the invention provides a method for expanding, within a non-adherent cell cluster, the number of cells capable of differentiating along a pancreatic lineage.

In one embodiment, the method comprises expanding the number of pdx1+ cells in an insulin-, non-adherent cell cluster.

In one embodiment, the method comprises expanding the number of pdx1− cells in an insulin−, non-adherent cell cluster, whereby said pdx1− cells differentiate to pdx1+ cells.

In one embodiment, the method for expanding the number of cells capable of differentiating along a pancreatic cell lineage comprises culturing cells in acidic media, whereby the cells receive an acid shock. In one embodiment, said acid shock comprises culturing cells in acidic media for at least 1 minute. In another embodiment, the method comprises culturing cells in acidic media for at least 2, 3, 4, 5, 6, 7, 8, 9, 10, 11, 12, 13, 14 or 15 minutes. In another embodiment, the method comprises culturing cells in acidic media for at least 15, 30, 45, 60, 90 or 120 minutes. In another embodiment, the method comprises culturing cells in acidic media for at least 2-24 hours. In still another embodiment, the method comprises culturing cells in acidic media for 24-48 hours.

In another embodiment, the method comprises culturing cells in acidic media in the presence of an FGF mitogen and an agent that increases intracellular cAMP.

In another embodiment, the method comprises culturing cells in acidic media in the presence of an FGF mitogen, an agent that increases intracellular cAMP and/or insulin and a corticosteroid.

In still another embodiment, the method comprises culturing cells in acidic media in the presence of an FGF mitogen, an agent that increases intracellular cAMP, insulin and a corticosteroid.

In any of the foregoing embodiments of this aspect of the present invention, the expansion medium includes follistatin and/or a follistatin-related protein (herein the term follistatin-based factors will be used generically to refer to follistatin and follistatin-related proteins). In one embodiment, the follistatin related protein includes inhibin or another related protein that negatively regulates activin via the same mechanism as follistatin (e.g., directly binding to activin). In another embodiment, the expansion medium includes a follistatin-related gene protein. In still another related embodiment, the expansion medium includes an inhibitor of activin. The invention contemplates the addition of one or more of the foregoing follistatin-based factors or inhibitors of activin at any point during the isolation or expansion protocol. Similarly, the invention contemplates the addition of one or more of the foregoing follistatin-based factors or inhibitors of activin at multiple points during the isolation or expansion protocols. Furthermore, the invention contemplates the addition of one or more of the foregoing follistatin-based factors or inhibitors of activin during the differentiation of expanded cells.

In any of the foregoing embodiments of this aspect of the present invention, the expansion medium includes exendin-4 and/or a GLP-1 analog (herein the term GLP-1 agonist will be used generically to refer to exendin-4, exendin-3, GLP-1, and other GLP-1 analogs including mimetics and modified or derivatized forms of any of the foregoing GLP-1 agonists). The invention contemplates the addition of one or more of the foregoing GLP-1 agonists at any point during the isolation or expansion protocol. Similarly, the invention contemplates the addition of one or more of the foregoing GLP-1 agonists at multiple points during the isolation or expansion protocols. Furthermore, the invention contemplates the addition of one or more of the foregoing GLP-1 agonists during the differentiation of expanded cells. Additionally, the invention contemplates the addition of one or more GLP-1 agonists and one or more follistatin-based factors at any step during the isolation, expansion, and/or differentiation of the cells.

In any of the foregoing embodiments of this aspect of the present invention, the FGF mitogen can be selected from any FGF polypeptide. In one embodiment, the FGF mitogen is selected from FGF-5, FGF-7, FGF-8, FGF- 10, FGF- 16, FGF-17 and FGF-18. In another embodiment, the FGF mitogen is selected from FGF-8, FGF-17 and FGF-18. In another embodiment, the FGF mitogen is selected from FGF-18.

In any of the foregoing embodiments of this aspect of the present invention, the agent that increases intracellular cAMP can be selected from any agent that elevates intracellular cAMP. In one embodiment, the agent is selected from CPT-cAMP, forskolin, Na-Butyrate, isobutyl methylxanthine, cholera toxin, 8-bromo-cAMP, dibutyrl-cAMP, dioctanoyl-cAMP, pertussis toxin, prostaglandins, colforsin, β-adrenergic receptor agonists, and cAMP analogs. In another embodiment, the agent is selected from forskolin.

In any of the foregoing embodiments of this aspect of the present invention, the corticosteroid can be selected from any corticosteroid. In one embodiment, the corticosteroid is selected from the group consisting of dexamethasone, hydrocortisone, cortisone, prednisolone, methylprednisolone, triamcinolone, and betamethasone.

In one embodiment, the insulin− cells are isolated from pancreatic tissue.

In another embodiment, the insulin− cells are isolated from duct or tubule tissue. In another embodiment, the duct or tubule tissue is selected from the group consisting of pancreatic duct, hepatic duct, kidney duct, kidney tubule (e.g., proximal tubule, distal tubule), bile duct, tear duct, lactiferous duct, ejaculatory duct, seminiferous tubule, efferent duct, cystic duct, lymphatic duct, and thoracic duct.

In another embodiment, the insulin− cells are stem cells selected from the group consisting of embryonic stem cells, fetal stem cells, and adult stem cells. In one embodiment, the adult stem cells are selected from the group consisting of neural stem cells, neural crest stem cells, pancreatic stem cells, skin-derived stem cells, cardiac stem cells, liver stem cells, endothelial stem cells, hematopoietic stem cells, and mesenchymal stem cells. In another embodiment, the adult stem cells are isolated from an adult tissue. In yet another embodiment, the stem cells are isolated from an adult tissue selected from the group consisting of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, and olfactory epithelium.

In a fifth aspect, the invention provides a method of differentiating substantially purified, insulin− cells to insulin+, glucose responsive cells following the initial expansion of pdx1+ cells within clusters of insulin− cells.

In a sixth aspect, the invention provides a composition of islet-like structures differentiated by any of the foregoing methods. Such islet-like structures may be differentiated following an initial expansion method to increase the pdx1+ cells within clusters of insulin− cells. In a preferred embodiment, the islet-like structures contain insulin+, glucose responsive cells. In another preferred embodiment, the islet-like structures additionally contain glucagon+ and somatostatin+ cells. In still another preferred embodiment, the glucagon+ and somatostatin+ cells are localized to the periphery of the islet-like structure.

In a seventh aspect, the invention provides a composition of insulin+, glucose responsive cells differentiated by any of the foregoing methods. Such insulin+, glucose responsive cells may be differentiated following an initial expansion method to increase the pdx1+ cells within clusters of insulin− cells

In an eighth aspect, the invention provides a composition of cell clusters expanded by the methods of the present invention to include an increased proportion of pdx1+ cells. In one embodiment, the cell clusters comprise at least 10-fold, 20-fold, 50-fold, 60-fold, 80-fold, or 100-fold more pdx1+ cells than observed in cell clusters which were not previously expanded by the methods of the present invention. In another embodiment, the cell clusters comprise at least 100-fold, 150-fold, 200-fold, 225-fold, 250-fold, 275-fold, 300-fold, or 500-fold more pdx1+ cells than observed in cell clusters which were not previously expanded by the methods of the present invention.

In a ninth aspect, the invention provides methods for treating a patient by transplanting a therapeutically effective amount of glucose responsive, insulin+ cells. In one embodiment, the glucose responsive, insulin+ cells comprise islet-like structures. In one embodiment, the patient is a human patient. In another embodiment, the patient has a condition characterized by an impaired responsiveness to glucose. Such conditions include diabetes, obesity, cancer, and pancreatic injury.

In another embodiment, the invention contemplates that the insulin+, glucose responsive cells may be administered either alone, or in combination with other therapeutic agents or regimens. Exemplary therapeutic agents and regimens include, but are not limited to, insulin, diet and exercise.

In a tenth aspect, the invention provides for the use of insulin+, glucose responsive cells in the manufacture of a medicament for treating a condition in a patient, wherein said condition is characterized by an inhibition in the ability of said patient's body to properly respond to glucose.

In one embodiment, the condition comprises diabetes. In another embodiment, the condition comprises an injury to or a disease of the pancreas. In another embodiment, the condition comprises an injury to or a disease of the β-cells of the pancreas.

In an eleventh aspect, the invention provides a method of priming a population of cells in culture, comprising culturing said cells in acidic media, thereby providing an acidic shock which primes said cells and thus promotes the ability of these cells to expand to pdx1+ cells.

In one embodiment, the acidic shock comprises culturing said cells in acidic media for at least one minute. In another embodiment, the acidic shock comprises culturing said cells in acidic media for at least 2, 3, 4, 5, 6, 7, 8, 9, 10, 11, 12, 13, 14, or 15 minutes. In another embodiment, the acidic shock comprises culturing said cells in acidic media for at least 15, 30, 45, 60, 90 or 120 minutes. In still another embodiment, the acidic shock comprises culturing said cells in acidic media for at least 2-24 hours.

In one embodiment, the cells are stem cells. In another embodiment, the stem cells are selected from the group consisting of embryonic stem cells, fetal stem cells, and adult stem cells. In another embodiment, the adult stem cells are selected from the group consisting of neural stem cells, neural crest stem cells, pancreatic stem cells, skin-derived stem cells, cardiac stem cells, liver stem cells, endothelial stem cells, hematopoietic stem cells, and mesenchymal stem cells. In another embodiment, the adult stem cells are isolated from an adult tissue. In yet another embodiment, the stem cells are isolated from an adult tissue selected from the group consisting of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, and olfactory epithelium.

In a twelfth aspect, the invention provides an improved method of dissociating a cluster of cells, comprising culturing the cluster of cells in the presence of Protease XXIII. In one embodiment, the cells are stem cells. In another embodiment, the stem cells are selected from the group consisting of embryonic stem cells, fetal stem cells, and adult stem cells. In another embodiment, the adult stem cells are selected from the group consisting of neural stem cells, neural crest stem cells, pancreatic stem cells, skin-derived stem cells, cardiac stem cells, liver stem cells, endothelial stem cells, hematopoietic stem cells, and mesenchymal stem cells. In another embodiment, the adult stem cells are isolated from an adult tissue. In yet another embodiment, the stem cells are isolated from an adult tissue selected from the group consisting of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, and olfactory epithelium.

In any of the foregoing aspects of the invention, except where specifically noted, expression of a given marker is meant to comprise the expression of a particular protein as measured by immunohistochemistry. For example, insulin+ or insulin− is meant to indicate that a given cell expresses insulin protein (+) or does not express insulin protein (−).

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning: A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No.: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Other features and advantages of the invention will be apparent from the following detailed description, and from the claims.

### DETAILED DESCRIPTION OF THE DRAWINGS

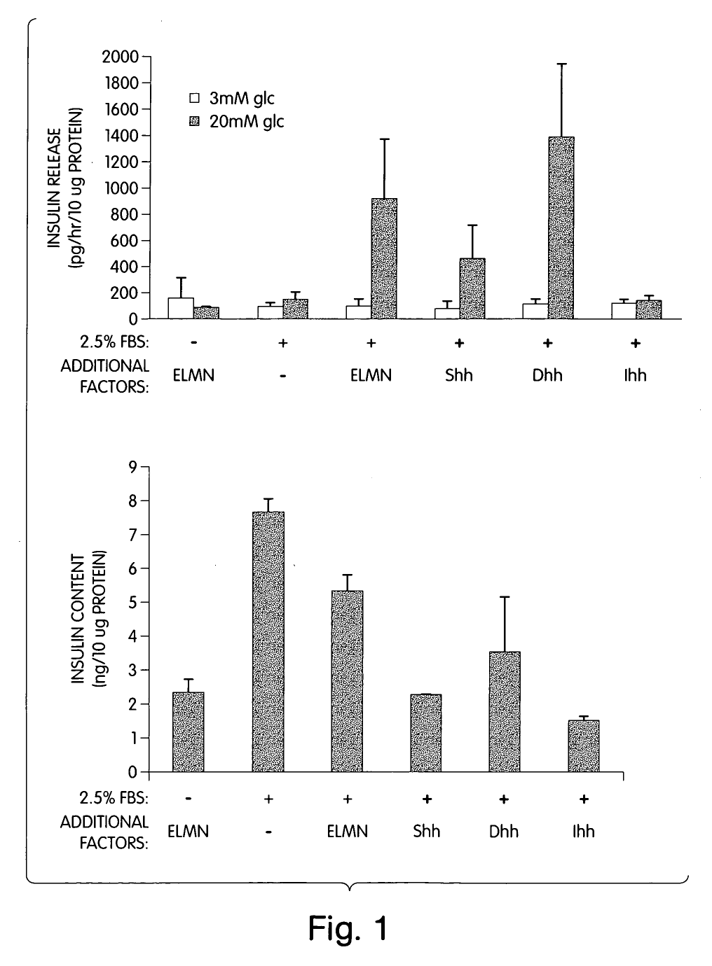

FIG. 1 shows that differentiated islet-like structures produced using the methods of the present invention are glucose responsive. Islet-like structures were differentiated, and cultured through step (f) in the presence of serum and either 3 mM or 20 mM glucose. The graph indicates that the islet-like structures respond to glucose by releasing insulin. Additionally, the media was supplemented with factors which appear to boost the responsiveness of the islet-like structures to glucose. These factors include the cocktail ELMN (exendin-4, leptin, malonyl CoA, nicotinamide), or hedgehog polypeptides (desert, Indian, and sonic). These factors may help prime the islet-like structures to respond to glucose. Alternatively, these factors may help to recapitulate signaling that occurs in the in vivo environment.

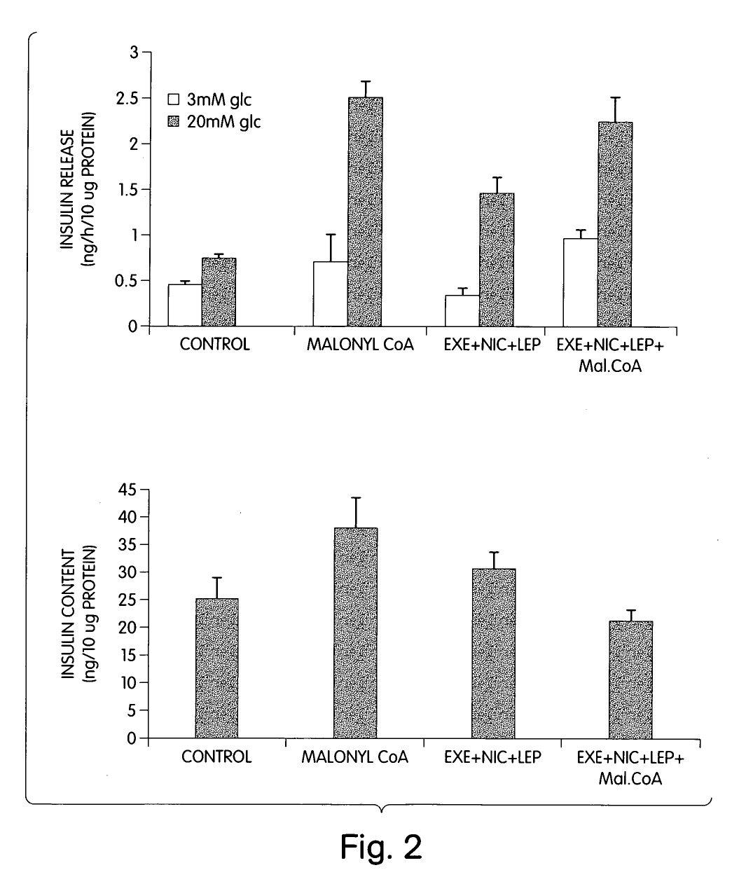

FIG. 2 shows that differentiated islet-like structures produced using the methods of the present invention are glucose responsive. Similar to the results summarized in FIG. 1, FIG. 2 demonstrates that the islet-like structures are glucose responsive, and that factors including malonyl CoA, exendin-4, nicotinamide, and leptin may help to further stimulate the responsiveness of the islet-like structures to glucose.

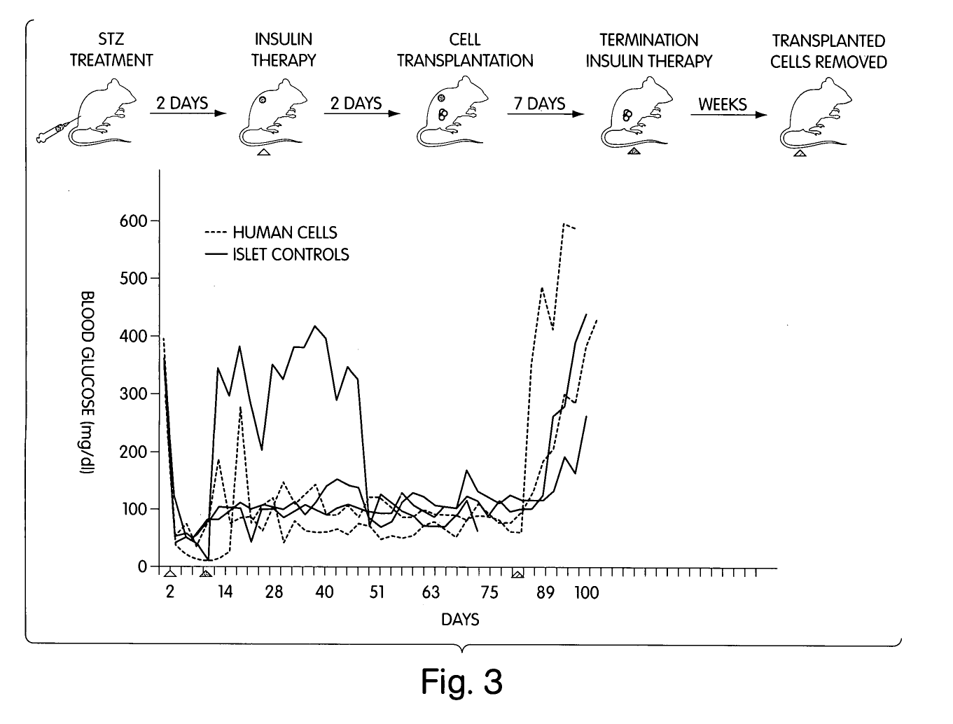

FIG. 3 shows that transplantation of in vitro differentiated, insulin+, glucose responsive human cells can successfully rescue normal blood glucose levels in STZ-treated diabetic mice. NOD-Scid female mice with normal blood glucose levels of 90-120 mg/dl were injected with a single dose of streptozotocin (STZ). Mice with a blood glucose level over 350 mg/dl on two consecutive days were implanted subcutaneously with a sustained release bovine insulin implant. Two days later, animals were transplanted with either rat islets or in vitro differentiated, insulin+ human cells. Insulin therapy delivered by the bovine implant was maintained for seven days after islet or human cell transplantation to ensure engraftment of the cells. Following removal of the bovine insulin implant, blood glucose levels normalized between 90-120 mg/ml for mice transplanted with rat islets (n=2/2) and for mice transplanted with in vitro differentiated, insulin+ human cells (n=2/3).

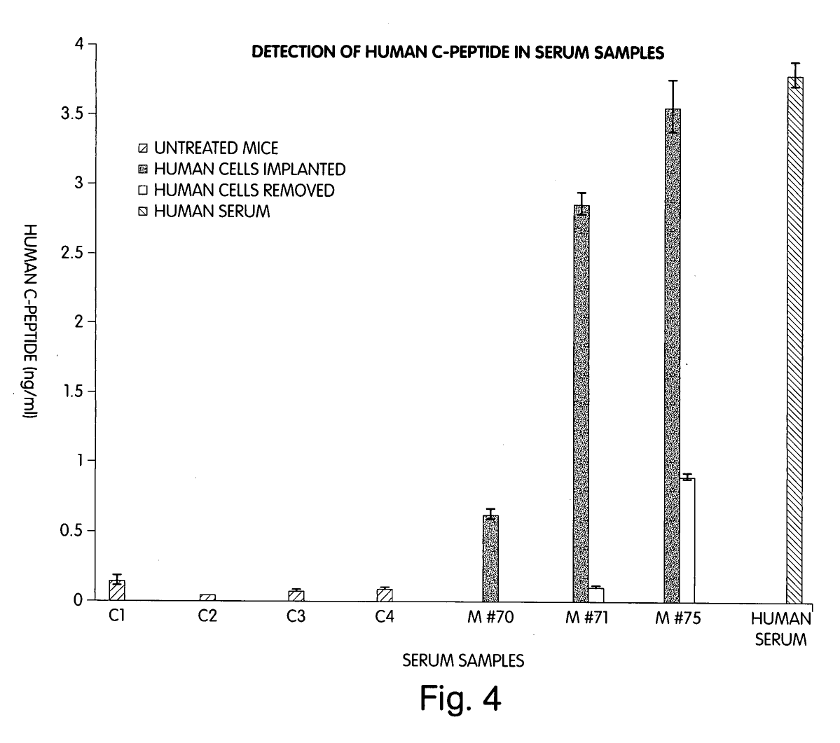

FIG. 4 shows the results of radioimmunoassay for human insulin C-peptide. Radioimmunoassays were performed six weeks after blood glucose values had stabilized to confirm the presence of secreted human insulin in mice transplanted with human cells. Non-fasting serum samples were obtained from control mice, mice transplanted with rat islets, and mice transplanted with in vitro differentiated insulin+ human cells. Analysis of a sample of human serum served as a positive control for the assay method. The graph shows that untreated mice test negative for human C-peptide, while mice transplanted with in vitro differentiated, insulin+, human cells test positive for human C-peptide.

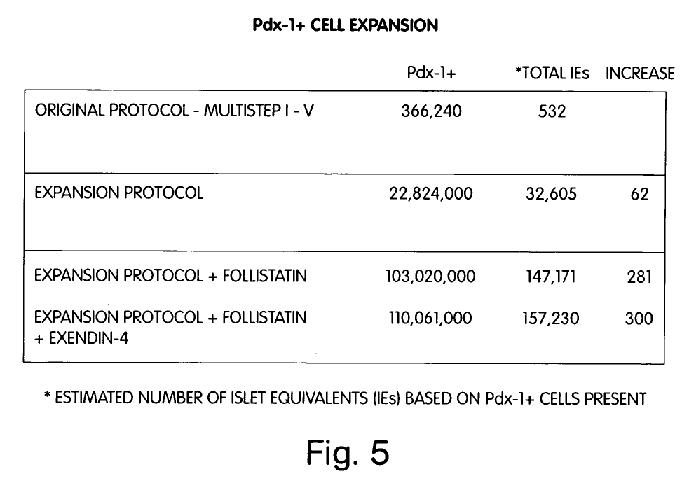

FIG. 5 summarizes experiments demonstrating the effectiveness of the expansion protocol (in the presence or absence of follistatin and/or exendin-4) in increasing both the number of pdx1+ cells and the total number of islet equivalents (IEs) in comparison to the multi-step differentiation protocol alone in the absence of the expansion protocol. Briefly, the use of the expansion protocol resulted in an approximately 62 fold increase in pdx1+ cells and total IEs in comparison to the use of the multistep differentiation protocol alone. Additionally, supplementation of the factors used in the expansion protocol with either follistatin or with a combination of follistatin and exendin-4 resulted in a 281 fold and 300 fold increase, respectively, in both pdx1+ cells and in total IEs.

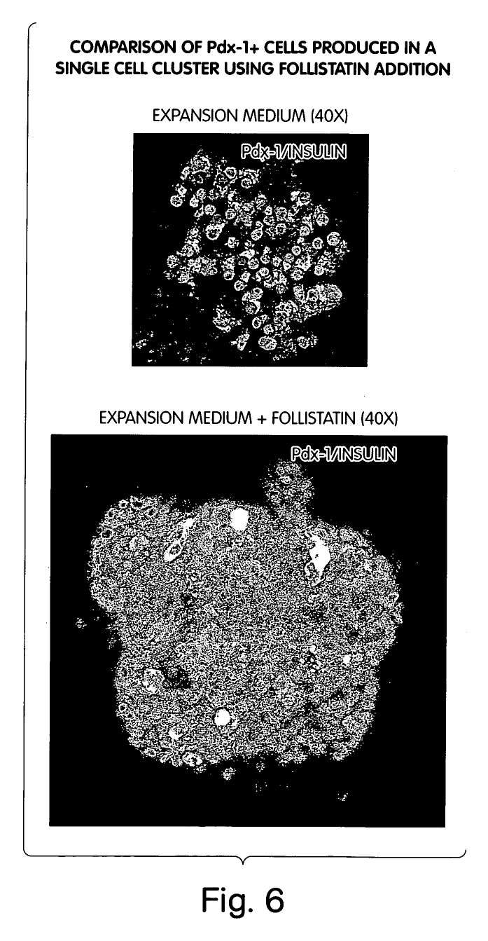

FIG. 6 shows a comparison of pdx1+ cells and insulin+ cells in cell clusters cultured under expansion conditions or under expansion conditions supplemented with follistatin. These results demonstrate that addition of follistatin to the expansion medium increased the number of pdx1+ cells in comparison to culture in expansion medium lacking follistatin.

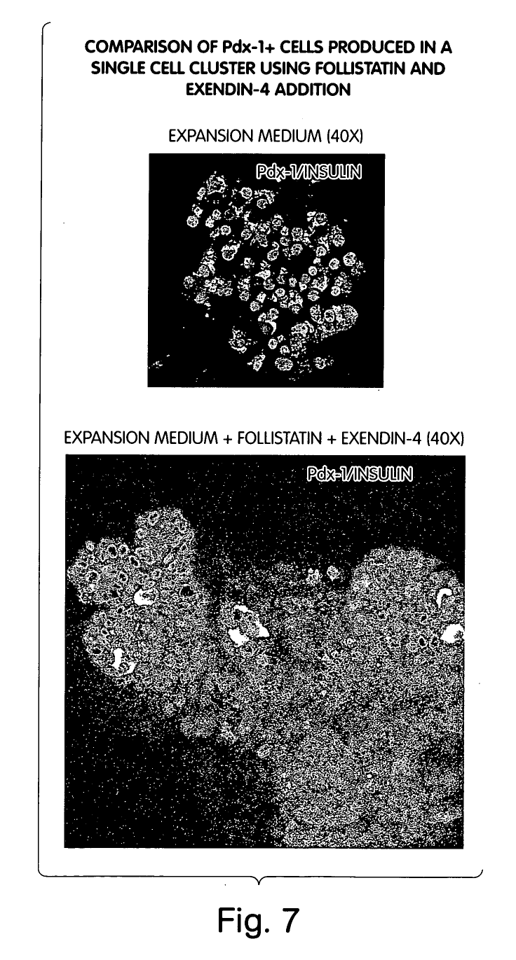

FIG. 7 shows a comparison of pdx1+ cells and insulin+ cells in cell clusters cultured under expansion conditions or under expansion conditions supplemented with follistatin and exendin-4. These results demonstrate that addition of follistatin to the expansion medium increased the number of pdx1+ cells in comparison to culture in expansion medium lacking follistatin.

### DETAILED DESCRIPTION OF THE INVENTION

(i) Definitions

For convenience, certain terms employed in the specification, examples, and appended claims are collected here.

The term “adherent matrix” refers to any matrix that promotes adherence of cells in culture (eg. fibronectin, collagen, laminins, superfibronectin). Exemplary matrices include Matrigel (Beckton-Dickinson), HTB9 matrix, and superfibronectin. Matrigel is derived from a mouse sarcoma cell line. HTB9 is derived from a bladder cell carcinoma line (U.S. Pat. No. 5,874,306).

As used herein the term “animal” refers to mammals, preferably mammals such as humans. Likewise, a “patient” or “subject” to be treated by the method of the invention can mean either a human or non-human animal.

“Differentiation” in the present context means the formation of cells expressing markers known to be associated with cells that are more specialized and closer to becoming terminally differentiated cells incapable of further division or differentiation. For example, in a pancreatic context, differentiation can be seen in the production of islet-like cell clusters containing an increased proportion of β-epithelial cells that produce increased amounts of insulin.

The term “progenitor cell” is used synonymously with “stem cell”. Both terms refer to an undifferentiated cell which is capable of proliferation and giving rise to more progenitor cells having the ability to generate a large number of mother cells that can in turn give rise to differentiated, or differentiable daughter cells. In a preferred embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors.

The term “embryonic stem cell” is used to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970).

The term “adult stem cell” is used to refer to any multipotent stem cell derived from non-embryonic tissue, including fetal, juvenile, and adult tissue. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture.

“Proliferation” indicates an increase in cell number.

The term “tissue” refers to a group or layer of similarly specialized cells which together perform certain special functions.

The term “pancreas” is art recognized, and refers generally to a large, elongated, racemose gland situated transversely behind the stomach, between the spleen and duodenum. The pancreatic exocrine function, e.g., external secretion, provides a source of digestive enzymes. Indeed, “pancreatin” refers to a substance from the pancreas containing enzymes, principally amylase, protease, and lipase, which substance is used as a digestive aid. The exocrine portion is composed of several serous cells surrounding a lumen. These cells synthesize and secrete digestive enzymes such as trypsinogen, chymotrypsinogen, carboxypeptidase, ribonuclease, deoxyribonuclease, triacylglycerol lipase, phospholipase A₂, elastase, and amylase.

The endocrine portion of the pancreas is composed of the islets of Langerhans. The islets of Langerhans appear as rounded clusters of cells embedded within the exocrine pancreas. Four different types of cells—α, β, δ, and φ—have been identified in the islets. The α cells constitute about 20% of the cells found in pancreatic islets and produce the hormone glucagon. Glucagon acts on several tissues to make energy available in the intervals between feeding. In the liver, glucagon causes breakdown of glycogen and promotes gluconeogenesis from amino acid precursors. The δ cells produce somatostatin which acts in the pancreas to inhibit glucagon release and to decrease pancreatic exocrine secretion. The hormone pancreatic polypeptide (PP) is produced in the φ cells. This hormone inhibits pancreatic exocrine secretion of bicarbonate and enzymes, causes relaxation of the gallbladder, and decreases bile secretion. The most abundant cell in the islets, constituting 60-80% of the cells, is the β cell, which produces insulin. Insulin is known to cause the storage of excess nutrients arising during and shortly after feeding. The major target organs for insulin are the liver, muscle, and fat-organs specialized for storage of energy.

The term “pancreatic duct” includes the accessory pancreatic duct, dorsal pancreatic duct, main pancreatic duct and ventral pancreatic duct. Serous glands have extensions of the lumen between adjacent secretory cells, and these are called intercellular canaliculi. The term “interlobular ducts” refers to intercalated ducts and striated ducts found within lobules of secretory units in the pancreas. The “intercalated ducts” refers to the first duct segment draining a secretory acinus or tubule. Intercalated ducts often have carbonic anhydrase activity, such that bicarbonate ion may be added to the secretions at this level. “Striated ducts” are the largest of the intralobular duct components and are capable of modifying the ionic composition of secretions.

The term “pancreatic progenitor cell” refers to a cell which can differentiate into a cell of pancreatic lineage, e.g. a cell which can produce a hormone or enzyme normally produced by a pancreatic cell. For instance, a pancreatic progenitor cell may be caused to differentiate, at least partially, into α, β, δ, or φ islet cell, or a cell of exocrine fate. The pancreatic progenitor cells of the invention can also be cultured prior to administration to a subject under conditions which promote cell proliferation and differentiation. These conditions include culturing the cells to allow proliferation in vitro at which time the cells can be made to form pseudo islet-like aggregates or clusters and secrete insulin, glucagon, and somatostatin.

The term “islet-like structures” refers to the clusters of cells derived from the methods of the invention which take on both the appearance of pancreatic islets, as well as the function. Such functions include the ability to respond to glucose. The islet-like structures of the invention are distinct from many of those previously cultured using other methods because they recapitulate the spatial relationship among the various cell types (i.e., somatostatin+ and glucagon+ cells are oriented toward the periphery of the islet). Additionally, the islet-like structures of the invention contain the insulin+, somatostatin+ and glucagon+ cells in approximately the same ratios as found endogenously in the pancreas.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the term “substantially pure” refers to a population of cells that contain fewer than about 20%, more preferably fewer than about 10%, most preferably fewer than about 5%, of lineage committed cells. In the context of the present invention, a lineage committed cell expresses at least one of the following markers of differentiated endocrine cells: insulin, somatostatin, or glucagons.

The term “non-adherent sphere” refers to the ability of the progenitor cells of the invention to proliferate in clusters. The cells are adherent to one another, but tend not to adhere to standard culture vessels. However, the cells will adhere when plated upon or cultured in the presence of an adherent substratum.

As used herein, “hedgehog polypeptide” refers to a polypeptide that is a member of the hedgehog family based on sequence, structure, and functional characteristics. Such functional characteristics include the ability to stimulate signaling through the hedgehog signaling pathway and the ability to bind the receptor patched. Hedgehog polypeptides are well known in the art, and are described for example in PCT publication WO95/18856 and WO96/17924 (hereby incorporated by reference in there entirety).

As used herein, “hedgehog therapeutic” refers to polypeptides, nucleic acids, and small molecules that stimulate or agonize hedgehog signaling. Exemplary hedgehog therapeutics include hedgehog polypeptides, small molecules which bind patched extracellularly and mimic hedgehog signaling, small molecules which bind smoothened, and small molecules which bind a protein involved in the intracellular tranduction of hedgehog signaling. Hedgehog therapeutics which stimulate or potentiate hedgehog signaling are also referred to as hedgehog agonists.

As used herein, “islet equivalents” or “IEs” is a measure used to compare total insulin content across a population or cluster of cells. An islet equivalent is defined based on total insulin content and an estimate of cell number which is typically quantified as total protein content. This allows standardization of the measure of insulin content based on the total number of cells within a cell cluster, culture, sphere, or other population of cells. The standard rat and human islet is approximately 150 μm in diameter and contains 40-60 ng insulin/μg of total protein. On average, human islet-like structures differentiated by the methods of the present invention contain approximately 50 ng insulin/pg of total protein.

(ii) Exemplary Embodiments

gp130 agonists: A family of cytokines has been identified which are characterized on the basis of signaling through the common signal transducer gp130 (Wijdenes et al. (1995) European Journal of Immunology 25: 3474-3481). This family of cytokines includes IL-6, IL-11, ciliary neurotrophic factor (CNTF), leukemia inhibitory factor (LIF), oncostatin M (OSM), and cardiotrophin-1. These factors are known to have a variety of roles. For example, LIF is commonly used to help promote the proliferation of embryonic stem cells, and additionally has been demonstrated to trigger proliferation in myoblasts, primordial germ cells, and some endothelial cells (Taupin et al. (1998) International Review of Immunology 16: 397-426). Cardiotrophin-1 induces cardiac myocyte hypertrophy in vitro, and also induces a liver acute phase response (Peters et al. (1995) FEBS Letter 372: 177-180). The effects of cardiotrophin-1 on rat hepatic cells is similar to that of LIF, and both cardiotrophin-1 and LIF have a more pronounced response than either oncostatin M or IL-6 in this system (Peters et al., supra).

More recent studies have demonstrated that cardiotrophin-1 has a wide range of effects in vivo when administered to mice where cardiotrophin-1 stimulates growth of heart, liver, kidney, and spleen tissue (Jin et al. (1996) Cytokine 8: 920-926). Additionally, two reports indicate that cardiotrophin-1 promotes neuronal survival, including the survival of dopaminergic neurons (Oppenheim et al. (2001) Journal of Neuroscience 21: 1283-1291; Pennica et al. (1995) Journal of Biological Chemistry 270: 10915-10922).

Clearly, gp130 agonists have a variety of roles in the development of many different systems. Their function in the methods of this invention has not been conclusively demonstrated, however, one possible role for the gp130 agonist is to promote cellular survival. To that end, it is expected that other gp130 agonists can functionally substitute for cardiotrophin-1 in the methods of the invention. The gp130 agonists may or may not function with equivalent potency, and the optimal gp130 agonist may vary, for example, according to the source of progenitor cells.

FGF family members: The FGF family of growth factors encompasses a large family of molecules implicated in cell patterning, proliferation, differentiation, and survival in a wide range of tissues. There are currently 20 identified mammalian FGFs, and these are expressed throughout embryonic and adult development, as well as in many pathological conditions.

There are many examples in the literature for the functional activity of various FGF family members. For example, FGF-5 or FGF-18 rescue photoreceptor cell death in two mice models of retinal degeneration (Green et al. (2001) Mol Ther 3: 507-515), FGF signaling is required for the proliferation and patterning of progenitor cells in the developing anterior pituitary (Norlin et al. (2000) Mechanisms of Development 96: 175-182), and a regulated gradient of FGF-8 and FGF-17 regulates proliferation and differentiation of midline cerebellar structures (Xu et al. (2000) Development 127: 1833-1843).

The methods of the present invention may employ any FGF family member, although it is anticipated that the various FGF family members will have differential efficacies in the claimed methods. We have examined the usefulness of FGF family members in both the differentiation methodologies exemplified herein, as well as in the methods of expanding pdx1+ cells prior to their differentiation. Accordingly, the present invention contemplates the use of any of these FGF family members during the methods of expansion and/or differentiation described in detail in the present application. Similarly the present invention contemplates embodiments in which multiple FGF family members are used during the expansion and/or differentiation methods described herein (e.g., two or more FGF family members are used at a particular step during the differentiation of the cells to insulin+, glucose responsive cells). Additionally, the present invention. contemplates embodiments wherein one or more FGF family member is used during both the expansion and differentiation of a particular culture or cluster of cells although both methods need not employ the same FGF family member.

Preferred FGF polypeptides are encoded by nucleic acids comprising an amino acid sequence at least 60% identical, more preferably 70% identical, and most preferably 80% identical with a vertebrate FGF polypeptide, or bioactive fragment thereof. Nucleic acids which encode polypeptides at least about 85%, more preferably at least about 90% or 95%, and most preferably at least about 98-99% identical with a vertebrate FGF polypeptide, or bioactive fragments thereof, are also within the scope of the invention. Bioactive fragments of FGF can be readily identified by, (a) the ability to bind an FGF receptor (there are currently 4 identified mammalian FGF receptors).

Functional analysis suggests that FGF-8/17/18 constitute a sub-group within the FGF family (Reifers et al. (2000) Mechanisms of Development 99: 39-49). In another embodiment, preferred FGF polypeptides are encoded by nucleic acids comprising an amino acid sequence at least 60% identical, more preferably 70% identical, and most preferably 80% identical with a vertebrate FGF-8, FGF-17, or FGF-18 polypeptide, or bioactive fragment thereof. Nucleic acids which encode polypeptides at least about 85%, more preferably at least about 90% or 95%, and most preferably at least about 98-99% identical with a vertebrate FGF-8, FGF-17, or FGF-18 polypeptide, or bioactive fragments thereof, are also within the scope of the invention. Bioactive fragments of FGF can be readily identified by, (a) the ability to bind an FGF receptor (there are currently 4 identified mammalian FGF receptors).

In addition, recent evidence suggests that FGF-7 may be particularly useful in stimulating pancreatic progenitor cells (Elghazi et al. PNAS 99: 3884-3889). Accordingly in another embodiment, the present invention contemplates that FGF polypeptides at least 60%, 70%, 75%, 80%, 85%, 90%, 95%, 99%, or identical to FGF-7 may be useful in the methods of the present invention.

Although any FGF family member may be used to practice the methods of this invention, there is evidence that suggests that FGF-18 is a good candidate to possess preferred activity in these methods. FGF-18 is expressed in the liver and pancreas, and ectopic expression of FGF-18 in mice induces proliferation in a variety of tissues. Specifically, FGF-18 expression induced significant proliferation in the liver and small intestines (Hu et al. (1998) Molecular and Cellular Biology 18: 6063-6074). Nevertheless, given the overlapping function of many FGF family members, the present invention contemplates the use of any of a number of FGF family members or combinations of family members in either the expansion or differentiation of insulin− cells and cell spheres to insulin+, glucose responsive cells and islet-like structures.

Hedgehog family members: Members of the hedgehog family of signaling molecules mediate many important short- and long-range patterning processes during invertebrate and vertebrate development. In the fly, a single hedgehog gene regulates segmental and imaginal disc patterning. In contrast, in vertebrates, a hedgehog gene family is involved in the control of left-right asymmetry, polarity in the CNS, somites and limb, organogenesis, chondrogenesis and spermatogenesis.

The vertebrate family of hedgehog genes includes at least four members, e.g., paralogs of the single Drosophila hedgehog gene. Exemplary hedgehog genes and proteins are described in PCT publications WO 95/18856 and WO 96/17924. Three of these members, herein referred to as Desert hedgehog (Dhh), Sonic hedgehog (Shh) and Indian hedgehog (Ihh), apparently exist in all vertebrates, including fish, birds, and mammals. A fourth member, herein referred to as tiggie-winkle hedgehog (Thh), appears specific to fish. Desert hedgehog (Dhh) is expressed principally in the testes, both in mouse embryonic development and in the adult rodent and human; indian hedgehog (Ihh) is involved in bone development during embryogenesis and in bone formation in the adult; and, Shh, which as described above, is primarily involved in morphogenic and neuroinductive activities. Despite the different roles fulfilled by the hedgehog family members during normal development, they are all capable of performing the same functions. Recent studies by Pathi and colleagues demonstrate that sonic hedgehog, desert hedgehog, and indian hedgehog all bind the receptor patched with the same kinetics. Additionally, the three hedgehog family members affect cell fate and behavior in the same way, albeit with differing potencies in a range of cell and tissue based assays (Pathi et al. (2001) Mechanisms of Development 106: 107-117).

The present methods employ steps including contacting cells with a hedgehog polypeptide. Without wishing to be bound by any particular theory, the result of contacting cells with a hedgehog polypeptide may be to activate hedgehog signaling in the cells and thus affect cell growth, proliferation, patterning, differentiation, and/or survival. Preferred hedgehog polypeptides are encoded by nucleic acids comprising an amino acid sequence at least 60% identical, more preferably 70% identical, and most preferably 80% identical with a vertebrate hedgehog polypeptide, or bioactive fragment thereof. Nucleic acids which encode polypeptides at least about 85%, more preferably at least about 90% or 95%, and most preferably at least about 98-99% identical with a vertebrate hedgehog polypeptide, or bioactive fragments thereof, are also within the scope of the invention. Bioactive fragments of hedgehog can be readily identified by, (a) the ability to bind the hedgehog receptor patched, (b) the ability to activate hedgehog signal transduction which can be assessed by, for example, transcription of hedgehog target genes. Particularly preferred hedgehog nucleic acids and polypeptides for use in the subject methods are at least 60%, 70%, 80%, 85%, 90%, 95%, or greater than 95% identical to human Sonic, human Desert, or human Indian hedgehog. Hedgehog polypeptides or active fragments thereof may be modified to include, for example, one or more hydrophobic moieties (Pepinsky et al. (1998) Journal of Biological Chemistry 273: 14037-45; Porter et al. (1996) Science 274: 255-9).

Additionally, one of skill in the art will recognize that if the function of contacting cells with a hedgehog polypeptide is to stimulate hedgehog signaling, then this can also be accomplished by contacting cells with other hedgehog therapeutic agents (i.e., hedgehog agonists). Such hedgehog therapeutics may stimulate hedgehog signaling by impinging upon the hedgehog signaling pathway at any point in the pathway. One of skill will recognize that such hedgehog therapeutics include nucleic acids, polypeptides, and small molecules that stimulate hedgehog signaling by acting at any point in the hedgehog pathway. Exemplary hedgehog therapeutics include small molecules that bind to patched and simulate hedgehog mediated signaling and small molecules that stimulate hedgehog signaling downstream of patched, thus by-passing the need to relieve patched mediated repression of hedgehog signaling. The methods of the present invention include contacting cells with a hedgehog polypeptide and one or more hedgehog therapeutics, or contacting cells with one or more hedgehog therapeutics (in the absence of a hedgehog polypeptide).

Feeder Layers: In aspects of the present invention, the method includes a step wherein the spheres are cultured on an adherent substratum. Without wishing to be bound be a particular theory, the substratum may secrete inductive factors and thus deliver a high local concentration of particular factors. The substratum also appears to provide a further purification of the desired progenitor cells. During culture of the spheres on the substratum, cells are observed to migrate out of the sphere and adhere to the substratum. Thus, the step of culturing the spheres on an adherent substratum may provide both inductive signals, as well as offer a means to further enrich for the desired cells.

Many types of adherent matrices/substratum can be used. In one embodiment, the spheres are cultured on a Matrigel layer. Matrigel (Collaborative Research, Inc., Bedford, Mass.) is a complex mixture of matrix and associated materials derived as an extract of murine basement membrane proteins, consisting predominantly of laminin, collagen IV, heparin sulfate proteoglycan, and nidogen and entactin, and was prepared from the EHS tumor (Kleinman et al, (1986) Biochemistry 25: 312-318). Other such matrixes can be provided, such as Humatrix. Likewise, natural and recombinantly engineered cells can be provided as feeder layers to the instant cultures.

In another embodiment, the culture vessels are coated with one or more extra-cellular matrix proteins including, but not limited to, fibronectin, superfibronectin, laminin, collagen, and heparin sulfate proteoglycan.

cAMP Elevating Agents: As described in detail herein, we have examined the usefulness of utilizing cAMP elevating agents in the expansion and/or differentiation methods of the present invention. In certain embodiments, the culture is contacted with the cAMP elevating agent forskolin. Similarly, in other embodiments, the culture is contacted with one or more cAMP elevating agents, such as 8-(4-chlorophenylthio)-adenosine-3′:5′-cyclic-monophosphate (CPT-cAMP) (see, for example, Koike. (1992) Prog. Neuro-Psychopharmacol. and Biol. Psychiat 16: 95-106), CPT-cAMP, forskolin, Na-Butyrate, isobutyl methylxanthine (IBMX), cholera toxin (see Martin et al. (1992) J. Neurobiol 23: 1205-1220), 8-bromo-cAMP, dibutyryl-cAMP and dioctanoyl-cAMP (e.g., see Rydel et al. (1988) PNAS 85: 1257).

As described in further detail below, it is contemplated that the subject methods can be carried out using cyclic AMP (cAMP) agonists. In yet other embodiments, the invention contemplates the in vivo administration of cAMP agonists to patients which have been transplanted with pancreatic tissue, as well as to patients which have a need for improved pancreatic performance, especially of glucose-dependent insulin secretion.

In light of the present disclosure, it will be apparent to those in the art that a variety of different small molecules can be readily identified, for example, by routine drug screening assays, which upregulate cAMP -dependent activities. For example, the subject method can be carried out using compounds which may activate adenylate cyclase including forskolin (FK), cholera toxin (CT), pertussis toxin (PT), prostaglandins (e.g., PGE-1 and PGE-2), colforsin and β-adrenergic receptor agonists. β-Adrenergic receptor agonists (sometimes referred to herein as “β-adrenergic agonists”) include albuterol, bambuterol, bitolterol, carbuterol, clenbuterol, clorprenaline, denopamine, dioxethedrine, dopexamine, ephedrine, epinephrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, prenalterol, procaterol, protokylol, reproterol, rimiterol, ritodrine, soterenol, salmeterol, terbutaline, tretoquinol, tulobuterol, and xamoterol.

Compounds which may inhibit cAMP phosphodiesterase(s), and thereby increase the half-life of cAMP, are also useful in the subject method. Such compounds include amrinone, milrinone, xanthine, methylxanthine, anagrelide, cilostamide, medorinone, indolidan, rolipram, 3-isobutyl-1-methylxanthine (IBMX), chelerythrine, cilostazol, glucocorticoids, griseolic acid, etazolate, caffeine, indomethacin, theophylline, papverine, methyl isobutylxanthine (MIX), and fenoxamine.

Certain analogs of cAMP, e.g., which are agonists of cAMP, can also be used. Exemplary cAMP analogs which may be useful in the present method include dibutyryl-cAMP (db-cAMP), (8-(4)-chlorophenylthio)-cAMP (cpt-cAMP), 8-[(4-bromo-2,3-dioxobutyl)thio]-cAMP, 2-[(4-bromo-2,3-dioxobutyl)thio]-cAMP, 8-bromo-cAMP, dioctanoyl-cAMP, Sp-adenosine 3′:5′-cyclic phosphorothioate, 8-piperidino-cAMP, N⁶-phenyl-cAMP, 8-methylamino-cAMP, 8-(6-aminohexyl)amino-cAMP, 2′-deoxy-cAMP, N⁶,2′-O-dibutryl-cAMP, N⁶,2′-O-disuccinyl-cAMP, N⁶-monobutyryl-cAMP, 2′-O-monobutyryl-cAMP, 2′-O-monobutryl-8-bromo-cAMP, N⁶-monobutryl-2′-deoxy-cAMP, and 2′-O-monosuccinyl-cAMP.

Above-listed compounds useful in the subject methods may be modified to increase the bioavailability, activity, or other pharmacologically relevant property of the compound. For example, forskolin has the formula:

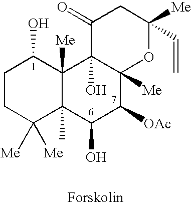

Modifications of forskolin which have been found to increase the hydrophilic character of forskolin without severely attenuating the desired biological activity include acylation of the hydroxyls at C6 and/or C7 (after removal of the acetyl group) with hydrophilic acyl groups. In compounds wherein C6 is acylated with a hydrophilic acyl group, C7 may optionally be deacetylated. Suitable hydrophilic acyl groups include groups having the structure —(CO)(CH₂)ₙX, wherein X is OH or NR₂; R is hydrogen, a C₁-C₄ alkyl group, or two Rs taken together form a ring comprising 3-8 atoms, preferably 5-7 atoms, which may include heteroatoms (e.g., piperazine or morpholine rings); and n is an integer from 1-6, preferably from 1-4, even more preferably from 1-2. Other suitable hydrophilic acyl groups include hydrophilic amino acids or derivatives thereof, such as aspartic acid, glutamic acid, asparagine, glutamine, serine, threonine, tyrosine, etc., including amino acids having a heterocyclic side chain. Forskolin, or other compounds listed above, modified by other possible hydrophilic acyl side chains known to those of skill in the art may be readily synthesized and tested for activity in the present method.

Similarly, variants or derivatives of any of the above-listed compounds may be effective as cAMP agonists in the subject method. Those skilled in the art will readily be able to synthesize and test such derivatives for suitable activity.

In certain embodiments, it may be advantageous to administer two or more of the above cAMP agonists, preferably of different types. For example, use of an adenylate cyclase agonist in conjunction with a cAMP phosphodiesterase antagonist may have an advantageous or synergistic effect.

The present invention contemplates the use of any of these cAMP elevating agents during the methods of expansion and/or differentiation described in detail in the present application. Similarly the present invention contemplates embodiments in which multiple cAMP elevating agents are used during the expansion and/or differentiation methods described herein (e.g., two or more cAMP elevating agents are used at a particular step during the differentiation of the cells to insulin+, glucose responsive cells). Additionally, the present invention contemplates embodiments wherein one or more cAMP elevating agent is used during both the expansion and differentiation of a particular culture or cluster of cells although both methods need not employ the same cAMP elevating agent(s).

Corticosteroids: The present methods contemplate that members of the subclass of steroids referred to as corticosteroids are useful in expanding the number of cells within non-adherent clusters of insulin− cells that are able to differentiate to form Pdx1+ cells (i.e., during the expansion method). The term steroid refers to any of a group of lipids that contain a hydrogenated cyclo-pentano-perhydrophenanthrene ring system. Exemplary classes of steroids include adrenocortical hormones (also known as corticosteroids), the gonadal hormones, cardiac aglycones, bile acids, sterols (such as cholesterol), toad poisons, and saponins.

Corticosteroids include any of the 21-carbon steroids which are endogenously elaborated by the adrenal cortex (excluding the sex hormones of adrenal origin) in response to adrenocorticotropic hormone (ACTH) released by the pituitary gland. Corticosteroids are typically subdivided based on their predominant biologic activity into glucocorticoids and mineralocorticoids. Generally glucocorticoids affect fat, carbohydrate, and protein metabolism while mineralocorticoids influence electrolyte and water balance, however these classifications are not absolute and some corticosteroids exhibit both types of activity. Exemplary corticosteroids include, but are not limited to, dexamethasone, hydrocortisone, cortisone, prednisolone, methylprednisolone, triamcinolone, and betamethasone.

Corticosteroids have been used in a clinical setting for hormonal replacement therapy, for suppression of ACTH secretion by the anterior pituitary, as an antineoplastic, as an antiallergic, as an anti-inflammatory, and as an immuno-suppressant.

The present invention contemplates the use of any of these corticosteroids during the method of expansion described in detail in the present application. Similarly the present invention contemplates embodiments in which multiple corticosteroids are used (e.g., two or more corticosteroids are used at a particular step during the expansion of the cells). When multiple corticosteroids are used, the invention contemplates their administration either at the same or different times. Additionally, the present invention contemplates that one or more corticosteroids can be administered at multiple time points during the expansion protocol. Without being bound by theory, one of skill in the art may wish to add additional corticosteroid(s) to the expansion medium to either boost the concentration of corticosteroid or to maintain a particular concentration of corticosteroid over the course of culture. This concept of boosting or refreshing culture medium over time is well known in the art of cell culture, and is often necessary given the finite half life of many proteins and small molecules. Accordingly, the present invention contemplates embodiments in which, following the initial addition of any of the particular protein or non-protein agents used to supplement the culture medium in the methods of the present invention, the agent is re-added to the culture medium.

Improved Methods of Dissociating Cell Clusters: In accordance with the methods of the present invention, cells are cultured as non-adherent clusters for a period of time, and then dissociated and plated. Although in theory cell clusters can be dissociated using any of a number of methods, many of these methods are relatively harsh and can cause damage to the cells and/or receptors on the cell surface that compromise the health and future proliferative and differentiative capabilities of these cells. Accordingly, the present invention offers a substantial improvement over the prior art by providing a method of dissociating clusters of cells which preserves the proliferative and differentiative capacity of the cells.

Without being bound by theory, we have discovered that Protease XXIII effectively dissociates cell clusters without compromising the health of the cells. Protease XXIII is also known in the art as Proteinase Type XXIII or Protease M Amano, and was originally purified from Aspergillus oryzae. It is commercially available from Sigma (www.sigmaaldrich.com), and we will use the terms Proteinase Type XXIII, Protease XXIII, and Protease M Amano interchangeably throughout to refer to this enzyme. One unit of commercially available enzyme is defined as the amount that will hydrolyze casein to produce color equivalent to 1.0 μmole of tyrosin per min at pH 7.5 at 37° C. The present invention further contemplates methods of dissociating cell clusters using an enzyme with substantially the same substrate specificity and activity as Protease XXIII.

The methods of the present invention contemplate that Protease XXIII can be used to dissociate clusters of cells including, but not limited to, clusters of stem cells. In one embodiment the clusters of stem cells can be selected from any of embryonic stem cells, fetal stem cells, and adult stem cells. The adult stem cells can be selected from any of neural stem cells, neural crest stem cells, pancreatic stem cells, skin-derived stem cells, cardiac stem cells, liver stem cells, endothelial stem cells, hematopoietic stem cells, and mesenchymal stem cells. Furthermore, the adult stem cells can be isolated from any adult tissue. In yet another embodiment, the stem cells are isolated from an adult tissue selected from any of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, and olfactory epithelium.

Expansion Method: The present invention provides a method for expanding (e.g., increasing) the number of cells in a cluster of cells which can differentiate to insulin+, glucose responsive cells. Thus, although the multi-step differentiation method described in detail in the present application results in the production of both insulin+, glucose responsive cells and islet-like structures containing a cellular organization consistent with that found in an endogenous islet, the expansion methodology outlined herein may be used to increase the efficiency of this process. Without being bound by theory, by expanding the number of cells within a culture or sphere of insulin− cells that are capable of differentiating to insulin+ glucose responsive cells, the expansion method can be used in combination with the multi-step differentiation method to increase the number of insulin+, glucose responsive cells obtainable from a given initial culture of insulin− cells.

Additionally, however, the present invention contemplates the use of the expansion method alone. The expansion method increases the number of cells within a culture or sphere of cells that are capable of differentiating to insulin+, glucose responsive cells. Such expanded cell populations can be assayed by an increase in the number of pdx1 expressing cells. Although not yet terminally differentiated to insulin+, glucose responsive cells, these expanded cells cultures or spheres may be used in screening assays to identify other factors useful in influencing terminal differentiation of pdx1+ cells (e.g., to insulin+, glucose responsive cells; to glucagon+ cells; to somatostatin+ cells, etc). Furthermore, such biased, expanded cells or clusters of cells can themselves form the basis of a therapeutic. Biased cells or cell clusters can be transplanted in vivo to a human or animal patient in need (e.g., a diabetic patient). Following transplantation, the biased cells could respond to local, in vivo signals and differentiate to insulin+, glucose responsive cells. Given that the expansion method appears to function to increase the proportion of cells capable of differentiating to a insulin+, glucose responsive cell, such biased cells may be more readily influenced by in vivo factors and the in vivo microenvironment and could provide an efficient cellular therapy.

As detailed in the examples, the expansion methods comprise culturing the cell clusters in media supplemented with certain factors. In addition, the method utilizes an acid pulse. By acid pulse is meant that the cells are cultured in acidic media for at least 1 minute. Without being bound by theory, one of skill in the art might initially believe that exposing cells to acidic media as detailed in the methods of the present invention would be detrimental to their proliferative and/or differentiative capacity. However, we now demonstrate that such an acidic pulse promotes the expansion of cells which can differentiate to insulin+, glucose responsive cells. This acid pulse may help to prime the cells and facilitate their responsiveness to factors that expand the population of pdx1+ cells within the culture or sphere of cells. Although the mechanism mediating this priming or biasing influence of the acid pulse is not know, one possibility is that this acid pulse helps to promote the synchronization of cells in a cluster of cells, and thus increase the number of cells entering S phase of the cell cycle. In this way, a greater proportion of the cells in culture are capable of responding to factors which expand the pdx1+ cells in the culture. Nevertheless and regardless of the underlying mechanism governing the utility of an acidic pulse in promoting the expansion of pdx1+ cells, the experiments outlined in the examples demonstrate such a utility. This despite any prevailing view in the art as to the detrimental effects of acidic conditions on cells in culture. Accordingly, the present invention provides methods of using an acid pulse to prime cells, and thus promote their responsiveness to factors which promote expansion of pdx1 expression within a culture of cells. The present invention further provides methods of using an acid pulse and other acidic culture conditions as part of a method of promoting the expansion of pdx1 expression in a culture of cells.

In one embodiment, the acid pulse is at least one minute, however, acid pulses of up to several days are also contemplated. When acid pulses are employed, then the acidic media may be supplemented with additional factors, as outlined in Example 6. When brief acid pulses are employed, the acidic media may be supplemented. However, we additionally note that when the media is changed from acidic media to neutral media, then this neutral media may also be supplemented with the additional factors. Thus, although the particular embodiment detailed in Example 6 involves the continued culture of the cells in acidic medium which is then supplemented with additional factors, the invention further contemplates the use of an acidic shock (in the presence or absence of additional factors) followed by a transfer of the cells to neutral pH which is then supplemented with the expansion factors (such as forskolin, FGF, etc).

The expansion method outlined in detail herein, aspects of which are typified in the examples, optionally involves the addition of one or more factors to the culture medium during one or more phases of the expansion protocol. Many of factors have been discussed in detail above. In addition to methods employing one or more of a cAMP elevating agent, an FGF, and/or a corticosteroid, the present invention contemplates expansion methods employing follistatin (or other follistatin-related factors) and/or exendin-4 (or other GLP-1 agonists) either alone or in combination with one or more of the expansion factors detailed in Example 6.

Follistatin-based Factors

As outlined in detail in the examples, the present invention contemplates methods employing addition of one or more follistatin-based factors (herein referred to interchangeably as follistatin-based factors or follistatin-related factors).

Follistatin is a secreted protein capable of influencing the fate of many diverse cell types including not only neuronal and epidermal cells, but also cells derived from the mesoderm and endoderm. Without being bound by theory, the function of follistatin is thought to be mediated, at least in part, by its activin inhibitory activity. Follistatin inhibits activin by physically interacting with activin protein (Phillips and de Kretser (1998) Front Neuroendocrinology 19: 287-322; Mather et al (1997) Proc Soc Exp Biol Med 215: 209-222).

Other proteins which possess the activin inhibitory activity of follistatin have been identified. Examples of these follistatin-based factors include follistatin-related gene protein and inhibin (Wankell et al. (2001) Journal of Endocrinology 171: 385-395; Schneyer et al. (2001) Mol Cell Endocrinol 180: 33-38; Gaddy-Kurten et al. (2002) Endocrinology 143: 74-83). Accordingly, the expansion methods of the present invention contemplate not only the addition of follistatin to the expansion medium, but also the addition of one or more follistatin-based factor.

The present invention contemplates the use of one or more follistatin-based factors during the method of expansion described in detail in the present application. Similarly the present invention contemplates embodiments in which multiple follistatin-based factors are used (e.g., two or more follistatin-based factors are used at a particular step during the expansion of the cells). When multiple follistatin-based factors are used, the invention contemplates their administration either at the same or different times. Additionally, the present invention contemplates that one or more follistatin-based factors can be administered at multiple time points during the expansion protocol. Without being bound by theory, one of skill in the art may wish to add additional follistatin-based factors to the expansion medium to either boost the concentration of follistatin-based factors or to maintain a particular concentration of follistatin-based factors over the course of culture. This concept of boosting or refreshing culture medium over time is well known in the art of cell culture, and is often necessary given the finite half life of many proteins and small molecules. Accordingly, the present invention contemplates embodiments in which, following the initial addition of any of the particular protein or non-protein agents used to supplement the culture medium in the methods of the present invention, the agent is re-added to the culture medium.

Additionally, the potential use of follistatin-based factors is not limited to the expansion methodology detailed herein. The present invention contemplates addition of follistatin-based factors during the initial isolation of cells from tissue (for example, during the initial isolation of cells from pancreatic or other ductal tissue). The present invention similarly contemplates the addition of follistatin-based factors during differentiation of cells to insulin+, glucose responsive cells. Follistatin-based factors may be used at any point during the multi-step differentiation protocol described herein and such factors may also be added during more than one step in the differentiation process. Additionally, the invention contemplates the use of follistatin-based factors during the differentiation of cells to insulin+, glucose responsive cells regardless of whether follistatin-based factors were used during the expansion of those cells and also regardless of whether those cells were previously expanded. Furthermore, in embodiments in which follistatin-based factors are used during both the expansion and differentiation of the cells, the invention contemplates methods in which the same follistatin-based factor or factors are used in both methods, as well as embodiments in which different follistatin-based factors are used for the expansion of the cells versus the differentiation of the cells.

GLP-1 Agonists

As outlined in detail in the examples, the present invention contemplates methods employing addition of one or more GLP-1 agonists. GLP-1 (glucagon-like peptide-1) is an insulinotropic hormone that exerts its action via interaction with the GLP-1 receptor. Several GLP-1 agonists have been identified including exendin-3, exendin-4, and GLP-1 analogs which have been modified to increase their stability and in vivo half-life (Thum et al. (2002) Exper Clin Endocrinol Diabetes 110: 113-118; Aziz and Anderson (2002) Journal of Nutrition 132: 990-995; Tourrel et al. (2002) Diabetes 51: 1443-1452; Egan et al. (2002) Journal of Clin Endocrinol Metab 87: 1282-1290; Peters et al. (2001) Journal of Nutrition 131: 2164-2170; Tourrel et al. (2001) Diabetes 50: 1562-1570; Doyle and Egan (2001) Recent Prog Horm Res 56: 377-399).

Accordingly, the expansion methods of the present invention contemplate not only the addition of exendin-4 to the expansion medium, but also the addition of one or more GLP-1 analogs.

The present invention contemplates the use of one or more GLP-1 analogs during the method of expansion described in detail in the present application. Similarly the present invention contemplates embodiments in which multiple GLP-1 analogs are used (e.g., two or more GLP-1 analogs are used at a particular step during the expansion of the cells). When multiple GLP-1 analog are used, the invention contemplates their administration either at the same or different times. Additionally, the present invention contemplates that one or more GLP-1 analogs can be administered at multiple time points during the expansion protocol. Without being bound by theory, one of skill in the art may wish to add additional GLP-1 analogs to the expansion medium to either boost the concentration of GLP-1 analogs or to maintain a particular concentration of GLP-1 analogs over the course of culture. This concept of boosting or refreshing culture medium over time is well known in the art of cell culture, and is often necessary given the finite half life of many proteins and small molecules. Accordingly, the present invention contemplates embodiments in which, following the initial addition of any of the particular protein or non-protein agents used to supplement the culture medium in the methods of the present invention, the agent is re-added to the culture medium.

Additionally, the potential use of GLP-1 analogs is not limited to the expansion methodology detailed herein. The present invention contemplates addition of GLP-1 analogs during the initial isolation of cells from tissue (for example, during the initial isolation of cells from pancreatic or other ductal tissue). The present invention similarly contemplates that addition of GLP-1 analogs during differentiation of cells to insulin+, glucose responsive cells. GLP-1 analogs may be used at any point during the multi-step differentiation protocol described herein and such factors may also be added during more than one step in the differentiation process. Additionally, the invention contemplates the use of GLP-1 analogs during the differentiation of cells to insulin+, glucose responsive cells regardless of whether GLP-1 analogs were used during the expansion of those cells and also regardless of whether those cells were previously expanded. Furthermore, in embodiments in which GLP-1 analogs are used during both the expansion and differentiation of the cells, the invention contemplates methods in which the same GLP-1 analog or analogs are used, as well as embodiments in which different GLP-1 analogs are used for the expansion of the cells versus their differentiation.

(iii) Methods of Treatment

The present invention also provides substantially pure glucose responsive, insulin+ cells which can be used therapeutically for treatment of various disorders associated with insufficient functioning of the pancreas. The invention further provides substanitally pure islet-like structures, which islet-like structures comprise insulin+, glucose responsive cells, which can be used therapeutically for treatment of various disorders associated with insufficient functioning of the pancreas.

To illustrate, the subject islet-like structures can be used in the treatment or prophylaxis of a variety of pancreatic disorders, both exocrine and endocrine. For instance, the islet-like structures can be transplanted subsequent to partial pancreatectomy, e.g., excision of a portion of the pancreas. Likewise, such cell populations can be used to regenerate or replace pancreatic tissue lost due to, pancreatolysis, e.g., destruction of pancreatic tissue, such as pancreatitis, e.g., a condition due to autolysis of pancreatic tissue caused by escape of enzymes into the substance. Since the islet-like structures generated using the methods of the invention have a ratio of cell types consistent with that found in the endogenous pancreas, and since those cell types are properly oriented with respect to each other (i.e., somatostatin+ and glucagon+ cells found at the periphery of the islet), they are likely to provide effective treatment for disorders effecting all or a portion of the pancreas.

The primary aim of treatment in both forms of diabetes mellitus is the same, namely, the reduction of blood glucose levels to as near normal as possible. Treatment of Type 1 diabetes involves administration of replacement doses of insulin. In contrast, treatment of Type 2 diabetes frequently does not require administration of insulin. For example, initial therapy of Type 2 diabetes may be based on diet and lifestyle changes augmented by therapy with oral hypoglycemic agents such as sulfonylurea. Insulin therapy may be required, however, especially in the later stages of the disease, to produce control of hyperglycemia in an attempt to minimize complications of the disease, which may arise from islet exhaustion.

More recently, tissue-engineering approaches to treatment have focused on transplanting healthy pancreatic islets, usually encapsulated in a membrane to avoid immune rejection. Three general approaches have been tested in animal models. In the first, a tubular membrane is coiled in a housing that contains islets. The membrane is connected to a polymer graph that in turn connects the device to blood vessels. By manipulation of the membrane permeability, so as to allow free diffusion of glucose and insulin back and forth through the membrane, yet block passage of antibodies and lymphocytes, normoglycemia was maintained in pancreatectomized animals treated with this device (Sullivan et al. (1991) Science 252: 718).

In a second approach, hollow fibers containing islet cells were immobilized in the polysaccharide alginate. When the device was place intraperitoneally in diabetic animals, blood glucose levels were lowered and good tissue compatibility was observed (Lacey et al. (1991) Science 254: 1782).

The islet-like structures and/or the differentiated, insulin+, glucose responsive cells of the invention represent an excellent potential treatment option for either type of diabetes. A therapeutically effective amount of the islet-like structures of the invention can be transplanted into a patient in need in order to improve proper glucose responsiveness. The islet-like structures can be simply transplanted into the patient, or can be transplanted using any of the above outlined methods which may help to improve the efficacy of the transplanted tissue. Moreover, the invention contemplates that transplantation of islet-like structures and/or differentiated cells may be combined with other therapies. For example, transplantation may be supplemented with administration of exogenous insulin. Furthermore, given the important role of autoimmunity in the etiology of type 1 diabetes, transplantation may be supplemented with administration of immunosuppressive agents.

In the treatment of any of the above mentioned conditions, the dosage (i.e., what constitutes a therapeutically effective amount of islet-like structures) is expected to vary from patient to patient depending on a variety of factors. The selected dosage level will depend upon a variety of factors including the specific condition to be treated, other drugs, compounds and/or materials used in combination with the particular transplant, the severity of the patient's illness, the age, sex, weight, general health and prior medical history of the patient, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon factors including the patient's age, sex, and the severity of their injury or disease.

In the case of the present invention, the pharmaceutical composition comprises insulin+, glucose responsive cells differentiated by the methods of the present invention and one or more pharmaceutically acceptable carriers or excipients. As outlined above, the pharmaceutical composition may be administered in any of a number of ways including, but not limited to, systemically, intraperitonially, directly transplanted, and furthermore may be administered in association with hollow fibers, tubular membranes, shunts, or other biocompatible devices or scaffolds. Additionally, the pharmaceutical composition of the present invention may comprise islet-like structures containing insulin+, glucose responsive cells differentiated by the methods of the present invention and one or more pharmaceutically acceptable carriers or excipients. As outlined above, the pharmaceutical composition may be administered in any of a number of ways including, but not limited to, systemically, intraperitonially, directly transplanted, and furthermore may be administered in association with hollow fibers, tubular membranes, shunts, or other biocompatible devices or scaffolds.

Furthermore, the present invention contemplates methods of treatment based no the administration of cells or cell clusters that have been expanded in culture to increase the proportion of pdx1+ cells. Such cells have been biased to enhance their ability to differentiate along a pancreatic lineage. Without bing bound by theory, such biased cells can be transplanted in vivo and may more readily respond to the in vivo micro-environment to give rise to insulin+, glucose responsive cells, as well as to other cell type required in the patient.

Accordingly, the present invention provides a pharmaceutical composition comprising cells or cell clusters that have been expanded in culture to enhance the number of pdx1+ cells, in accordance with the methods of the present invention, and one or more pharmaceutically acceptable carriers or excipients. As outlined above, the pharmaceutical composition may be administered in any of a number of ways including, but not limited to, systemically, intraperitonially, directly transplanted, and furthermore may be administered in association with hollow fibers, tubular membranes, shunts, or other biocompatible devices or scaffolds.

The term “treatment” is intended to encompass also prophylaxis, therapy and cure, and the patient receiving this treatment is any animal in need, including primates, in particular humans, and other mammals such as equines, cattle, swine and sheep; as well as poultry and pets in general.

Exemplification

The invention now being generally described, it will be more readily understood by reference to the following examples which are included merely for purposes of illustration of certain aspects and embodiments of the present invention, and are not intended to limit the invention.

### EXAMPLE 1

### Isolation of Purified Pancreatic Cells

An important step of the present method is the purification of cells from tissue. We provide an improved method which results in highly purified population of cells from ductal tissue, and can be used to purify cells from any ductal or tubule tissue. In the following example, cells were purified from pancreatic ductal epithelium.

The pancreas was dissected from the spleen and intestines of an adult rat, and care was taken to remove exterior fat and membranous tissue from the pancreas. The pancreas was dissected into 2 mm² pieces of tissue in 1×HBSS media containing magnesium and calcium. The tissue was rinsed in ice-cold 1×HBSS to remove excess blood cells and adipose tissue.

The tissue was then centrifuged at 1500 rpm for 5 minutes, the media aspirated, and the centrifuged tissue transferred to Liberase Solution (Roche). The tissue was incubated in Liberase Solution at 37° C. for 15 minutes, with shaking at 180 rpm. Following this step, approximately 90% of the supernatant was decanted into a conical tube containing 10% BSA. The remaining tissue pieces were rinsed with ice cold HBSS buffer containing soybean trypsin inhibitor (SBTI), this supernatant was also decanted into the BSA, and fresh ice cold Liberase solution was then added to the remaining tissue.

All of the total decanted supernatant was centrifuged for 5 minutes at 1500 rpm, the supernatant removed, and the pellet resuspended in 100 mL HBSS with magnesium and calcium. This step is repeated as necessary.

The volume of the isolated duct fragments was brought to 225 mL with HBSS containing magnesium and calcium, Dnasel and Aprotinin were added, and the samples were incubated at 37° C. for 20 minutes. Following this incubation, the samples were centrifuged at 1500 rpm for 5 minutes, the supernatant aspirated off, and the pellet resuspended in HBSS lacking magnesium and calcium. This step was repeated, and the resulting pellet resuspended in 1.06 g/mL Percoll.

A Percoll gradient was prepared by layering the Percoll/pellet suspension with 1.04, 1.03, and 1.02 g/mL Percoll, and the samples were centrifuged at 1970 rpm for 10 minutes. Following centrifugation, there should be three layers of cells visible, and an exocrine pellet.

Using this purification method, we isolated a population of cells from ductal tissue that are substantially free of insulin+ cells. We estimated that the isolated cells contain less than 1% of contaminating insulin+ cells. Thus, these cells can be characterized as insulin− when assayed immunocytochemically. Furthermore, the cells are negative for glut2, an additional marker consistent with differentiation along a pancreatic or β-cell fate. The cells are also negative for nestin protein, a marker typically correlated with some other stem cell populations.

### EXAMPLE 2

### Isolation of Purified Human Pancreatic Cells

Human pancreas was harvested from a heart beating donor (age 7-30 years) and preserved in University of Wisconsin (UW) solution for up to 24 hours. One human pancreas was asceptically removed from UW solution and trimmed of adipose tissue, spleen and intestine. The pancreas was then cut into 3-4 equal portions, and transferred to a sterile dish containing cold tissue mincing buffer (UW solution+0.2% BSA+0.625 mg/ml soybean trypsin inhibitor). The portions of pancreas were cut into smaller pieces, transferred to a conical tube, and centrifuged at 1500 rpm at 4° C. for 5 minutes. Following removal of the supernatant, the tissue was washed with digestion wash buffer (1× calcium/magnesium containing Hanks Balanced Salt Solution+0.125 mg/ml soybean trypsin inhibitor) and centrifuged again.

Following the second centrifugation step and removal of the supernatant, the cells were resuspended in 10 ml of Liberase HI enzyme solution, and then transferred to another bottle containing an additional 80 ml of Liberase HI enzyme solution. The bottle containing tissue +90 ml of Liberase HI enzyme solution was incubated at 37° C. in a water bath with a maximum shaking speed of 188 cycles/minute. The tissue was initially digested for 15 minutes. Following this first digestion step, the supernatant was decanted (leaving the tissue pieces in the original bottle) into a centrifuge tube containing 80 ml of 10% BSA to inhibit enzyme activity as the ducts are being released. The remaining tissue pieces were rinsed with ice cold HBSS buffer containing SBTI, this supernatant was also decanted into the BSA, and the remaining tissue pieces were resuspended in fresh ice cold Liberase HI enzyme. The above steps were repeated 2-10 times, as needed.

The decanted supernatant, which contains ducts liberated from the digested pancreas tissue, was centrifuged at 2000 rpm for 20 minutes at 4° C., and the pellets were immediately resuspended in 40 ml suspension buffer (0.2% BSA, 1× calcium/magnesium containing Hanks Balanced Salt Solution+0.125 mg/ml soybean trypsin inhibitor)+DNAse and incubated at room temperature for 10 minutes. Following DNAse treatment, the ducts were centrifuged at 2000 rpm at 4° C. for 10 minutes, and the pellets were resuspended gently in ice cold 1× calcium/magnesium containing Hanks Balanced Salt Solution.

The duct suspension was layered over a sucrose cushion and centrifuged at 2000 rpm at 4° C. for 10 minutes to facilitate the removal of lipids and cellular debris. Following removal of the supernatant, the pellet was resuspended gently in basal medium (DMEM/F12 containing 2% B-27, 2 mM GlutaMAX, 100 U/ml Pen/Strep, 8 mM HEPES) and then transferred to a new tube containing basal medium+DNAse.

At this point, the sample contains ducts as well contaminating exocrine tissue and islets. Since the exocrine tissue and islets are heavier than the ducts, the samples are further purified via gravity by allowing the exocrine tissue and islets to settle for 20 minutes at room temperature. The supernatant, which is enriched for ducts, was transferred to a fresh tube and centrifuged at 2000 rpm at 4° C. for 10 minutes. The supernatant was decanted, and the duct-containing pellet was resuspended in basal medium.

### EXAMPLE 3

### Improved Method for Differentiating Insulin+, Islet-like Structures

The insulin− cells isolated from ductal or tubule tissue were cultured in serum-free DMEM/F-12 containing 8 mM HEPES and 2% B-27 (Basal Media) supplemented with 10 ng/mL of the gp130 agonist human Cardiotrophin-1. The cells were cultured for 6-7 days during which time they formed non-adherent spheres. Although not wishing to be bound by any particular theory, the presence of cardiotrophin-1, or another gp130 agonist, may act as a survival factor in much the same may that exogenous LIF added to the culture media seems to promote the proliferation of human embryonic stem cells.

In the next step, the spheres were dissociated to single cells using Protease XXIII/EDTA, and cultured in Basal Media supplemented with 20 ng/mL FGF-18, 100 ng/mL Sonic hedgehog, and 2 ug/mL heparin. The cells were cultured for 6-7 days, and during this expansion phase they proliferate, and reaggregate to form non-adherent spheres. Without wishing to be bound by any particular theory, FGF family members are growth factors with known mitogenic properties, and FGF-18 is normally expressed in the liver and pancreas. It seems likely that other FGF family members would have similar results in this method, and it seems especially likely that FGF family members closely related to FGF-18 such as FGF-8 and FGF-17 would have behave similarly in this method. Similarly, Hedgehog family members are known to promote growth and proliferation in a wide range of cellular contexts, and the various hedgehog family members (sonic, desert, and Indian) behave similarly in a variety of biochemical and cellular assays (Thomas et al. (2000) Diabetes 49: 2039-2047; Thomas et al. (2001) Endocrinology 142: 1033-1040). Accordingly, although sonic hedgehog was used here, we believe that other hedgehog polypeptides can be used with similar results. In fact, since hedgehog polypeptides act by activating the hedgehog signaling pathway, we believe that other agents which agonize hedgehog signaling could be used with similar effects. Examples of such hedgehog agonists would include small organic molecules which mimic the effects of hedgehog by binding to the receptor patched, or small organic molecules which act on a downstream target of hedgehog signaling. Heparin is believed to increase the localization of FGF family members to the cell membrane.

In the next step, the spheres were cultured in Basal Medium supplemented with several growth factors for 6-7 days. In these experiments the media was supplemented with EGF, FGF-18, IGF-1, IGF-11, TGF-α, VEGF, sonic hedgehog, and heparin. Such a cocktail of growth factors has been used by others, and we believe that one of skill could readily select combinations of growth factors belonging to these growth factor families for optimal use in the present methods. During this stage, the cells show signs of differentiation along a pancreatic lineage as measured by expression of insulin. A low, but substantial percentage of cells within the spheres express insulin (approximately 10% of the cells in the sphere).

In the next step, the spheres were plated on coated tissue culture plastic. The cells were not dissociated and plated, rather the spheres are plated. In these experiments, the tissue culture plastic was coated with either superfibronectin or poly-L-ornithine. We observe that cells within the spheres adhere to the matrix and appear to crawl out of the sphere. Without wishing to be bound by any particular theory, this may help to enrich for cells within the sphere which differentiate along a pancreatic lineage. The spheres were cultured for 4-5 days in RPMI media, which contains a relatively high glucose concentration (11.1 mM), supplemented with 1-5% serum, PYY, HGF, and forskolin.

One of skill in the art will recognize that forskolin is a cAMP elevating agent. We believe that a wide range of cAMP elevating agents may be used, either alone or in combination, in the methods of the present invention.

In the final step, the media was removed, and the spheres were cultured for 4-5 days in CMRL media containing a relatively low glucose concentration (5 mM) and supplemented with 1-5% serum, exendin-4, leptin, and nicotinamide. A similar cocktail of factors has been used by others in the past to help influence final differentiative events in pancreatic development (Lumelsky et al. (2001) Science 292: 1389-1394). At this point, we observed a substantial enrichment of insulin+ cells in the spheres. We estimated that approximately 90% of the cells remaining in the spheres are insulin+. Additionally, we observe somatostatin+ and glucagon+ cells. Expression of these markers is observed at approximately the same percentage observed endogenously during pancreatic development. Of particular note, the somatostatin+ and glucagon+ cells were oriented toward the periphery of the spheres which can now be considered islet-like structures. The spatial relationship among the insulin+, somatostatin+ and glucagon+ cells is important because it recapitulates the spatial relationship among the cells that occurs endogenously in the pancreas.

### EXAMPLE 4

### The Islet-like Structures are Glucose-responsive

Although the formation of islet-like structures and the expression of markers of pancreatic differentiation are consistent with functional islet formation, the only way to confirm that the islet-like structures are indeed functional is to demonstrate that the cells are responsive to glucose. FIGS. 1 and 2 summarize experiments which demonstrated that the islet-like structures were responsive to glucose (shown here 3 mM glucose and 20 mM glucose).

After the complete differentiation protocol described in detail in Example 2, the islet-like structures were cultured in the presence of either 3 mM glucose or 20 mM glucose to assay for glucose-stimulated insulin release. Insulin release and total insulin content were measured using standard methods. Additionally, factors were added to the culture of islet-like structures. FIG. 1 summarizes results which indicated that the addition of hedgehog polypeptides (sonic, desert, or Indian) increased the responsiveness of the structures to high glucose. FIG. 2 summarizes results which indicated that the addition of pancreatic maturation factors including malonyl CoA, exendin-4, nicotinamide, and leptin increased the responsiveness of the structures to high glucose.

Without wishing to be bound by any particular theory, the addition of factors including pancreatic maturation factors and/or hedgehog polypeptides may help the islet-like structures to complete some final stages of maturation necessary for an optimal response to glucose. Alternatively, these factors may mimic some of the endogenous signaling that occurs in the pancreas during a glucose response.

For therapeutic purposes, it may be advantageous to culture islet-like structures in the presence of one or more of the above cited maturation factors prior to transplantation inorder to “prime” or ready the islet-like structures for optimal glucose responsiveness. However, it is also possible that these factors will be supplied by the cellular environment following transplantation, and thus any priming required so that the islet-like structures attain maximal and efficient glucose responsiveness may happen in vivo once the structures are transplanted.

### EXAMPLE 5

### Transplantation of In Vitro Differentiated, Insulin+ Human Cells

One important utility of the present methods for the in vitro differentiation of insulin+, glucose responsive cells and islet-like clusters is that such tissue may be transplanted in vivo. Transplantation of these cells and/or islet-like clusters represents an attractive treatment option for diabetes, as well as other conditions which result in a destruction of functional β-islets or disturbance in the ability to modulate blood glucose levels. The practicality of this approach was tested in a mouse model of diabetes—STZ treated mice. Such mice are characterized by severely elevated blood glucose levels. We demonstrate that transplantation of insulin+ human cells, differentiated by the methods of the present invention, restored normal blood glucose levels in these mice. Additionally, use of transplanted human cells allowed us to confirm that the improvement in blood glucose levels in treated mice was the result of human insulin (produced by the transplanted tissue).

The experimental scheme and results of this experiment are summarized in FIG. 3. Briefly, six week old, NOD-SCID female mice with normal blood glucose levels between 90-120 mg/dl received a single IP dose of streptozotocin (STZ). The majority of injected mice exhibited elevated blood glucose levels within 24 hours. Mice whose blood glucose level measured greater than 350 mg/dl for two consecutive days were used for further study. Such mice were implanted subcutaneously with a sustained release bovine insulin therapy implant (Lin Shin Inc.), and divided into three random groups: control, rat islet recipients, and in vitro differentiated human cell recipients. You will note that following transplantation of the bovine implant, the blood glucose levels of the mice return to normal. Two days after transplantation of the bovine implant, mice received a second transplantation of either rat islets or human insulin+ cells differentiated in vitro by the methods of the present invention. The rat or human cells were transplanted directly into the fourth mammary gland fat pad. Mice received approximately 400 islet equivalents of insulin producing cells (either rat or human) determined from cellular extracts of insulin a day prior to the transplantation. Control mice received no further treatment. Insulin therapy via the bovine implant was maintained for seven days after transplantation of the rat or human tissue to ensure in vivo engraftment and insulin production.

Seven days after transplantation of rat islets or human insulin+ cells differentiated in vitro by the methods of the present invention, the bovine implant was removed. In the absence of the bovine implant, insulin production in these animals must be supplied by the transplanted rat or human tissue. Following removal of the bovine implants, blood glucose levels transiently increased for approximately two days. The blood glucose levels then returned to a normal range between 90-120 mg/dl for mice transplanted with either rat islets (n=2/2) or in vitro differentiated human cells (n=2/3). These normal blood glucose levels were maintained for eight weeks. In contrast, control mice (those mice receiving no additional therapy following the bovine implant) experienced an immediate elevation in their blood glucose levels following removal of the implant.

The results summarized in FIG. 3 demonstrate that insulin+ cells, differentiated in vitro by the methods of the present invention, can be transplanted in vivo to restore normal blood glucose levels. However, we performed additional analysis to confirm that the transplanted human cells were indeed producing insulin. By measuring the presence of human insulin C-peptide in the serum of treated mice, these experiments confirmed that the restoration of normal blood glucose levels in treated mice was the result of insulin produced by the human cells.

FIG. 4 summarizes the results of these experiments which demonstrated that untreated mice test negative for human insulin C-peptide, as one would expect. In contrast, mice transplanted with insulin+ human cells differentiated in vitro test positive for human insulin C-peptide, and such a positive test result is dependent on the presence of transplanted human cells (i.e., the presence of human insulin C-peptide decreases rapidly upon removal of the transplanted human cells).

Briefly, the presence of human insulin C-peptide in the serum of treated mice was measured by radioimmunoassay six weeks after blood glucose values had stabilized. Serum samples were obtained from untreated mice, mice transplanted with rat islets, and mice transplanted with in vitro differentiated human cells. As shown in FIG. 4, untreated mice test negative for human insulin C-peptide. In contrast, mice transplanted with insulin+, human cells differentiated in vitro by the methods of the present invention test positive for human insulin C-peptide, and this positive result is dependent upon the presence of the human cells in the animal. Additionally, we confirmed that mice transplanted with rat islets also test negative for human insulin C-peptide.

### EXAMPLE 6

### Expansion of Cells Capable of Generating Insulin+, Glucose Responsive Cells

A significant limitation in the art of cell based therapies, such as stem cell based therapies, is the limiting number of cells which appear to possess the desired characteristics and can be readily isolated from a given tissue sample. Accordingly, a significant improvement applicable to a wide range of methods designed to differentiate progenitor cells along a particular path involves methods which expand the population of cells capable of responding to a given differentiation protocol to generate a differentiated cell or tissue of interest. The present expansion protocol addresses this need. We have identified an expansion method which increases the number of pancreatic progenitor cells obtainable from a given tissue sample, and thus increases the number of cells capable of responding to a differentiation protocol to produce insulin+, glucose responsive cells.

Pancreatic duct cells were isolated from human donor tissue, using the methods described in detail in Example 2. The cells were plated as non-adherent cell clusters in DMEM-F12 (pH 7.4), 2 mM glutamine, 1% penicillin-streptomycin, 2% B27 (Life Sciences Technologies) and 8 mM HEPES. Following 1-4 days in culture, the media was changed to DMEM-F12 (pH 6.9-7.1), 2 mM glutamine, 1% penicillin-streptomycin, and 2% B27 (Life Sciences Technologies). This media was supplemented with the following four factors: dexamethasone (10⁻⁷-10⁻⁹ M), forskolin (10 μM), insulin (20 μg/ml) and FGF-18 (20 ng/ml). The media was optionally supplemented with heparin which is often used to enhance the effects of FGF. The cells were cultured for a number of days in this supplemented media which was changed daily.

Following approximately one day in culture, Pdx1+ cells (a marker of pancreatic progenitor cells) began appearing on the surface of the non-adherent clusters. The size and number of Pdx1+ cells continues to increase for approximately 12 days. Following 8 to 12 days in culture under expansion conditions, non-adherent cell clusters containing an increased number of Pdx1+ cells were subjected to a differentiation protocol to produce insulin+, glucose responsive cell clusters. We note that this expansion protocol also resulted in the production of Pdx1+ cell clusters in cultures of mouse embryonic stem cells, and may represent a general method of biasing cells along a pancreatic lineage.

Additional experiments assessed the relative contribution of the various components of the expansion protocol in generating Pdx1+ cells. Expansion is facilitated by the acidic culture conditions. Although we observed an increase in Pdx-1+ cells when non-adherent clusters were cultured in media maintained at pH 7.2-7.4, the emergence of Pdx-1+ cells was enhanced under acidic culture conditions (approximately pH 6.9-7.1). Furthermore, the invention contemplates that the emergence of Pdx-1+ cells can be enhanced by culturing the cells under acidic culture condition of approximately pH 5.0-7.2).

Although the maximum effect on cell expansion occurred in the presence of acidic medium supplemented with dexamethasone, an agent which increases intracellular cAMP, insulin and an FGF mitogen, we observed expansion of Pdx-1+ cells when the media was supplemented with only a subset of these factors. Specifically, the addition to the culture medium of an FGF mitogen and an agent which increases intracellular cAMP (i.e., a cAMP elevating agent) appears sufficient to produce an increase in the number of Pdx1+ cells. The invention further contemplates supplementation of the culture medium with the following concentration of factors: dexamethasone (10⁻⁵M-10⁻¹⁰M), forskolin (1-50 μM), insulin (5-200 μg/ml), and FGF (1-200 ng/ml).

### EXAMPLE 7

### Differentiation of Non-Adherent Clusters Previously Expanded in Culture

Cells were expanded in culture for 8-12 days, as described in Example 6. Following expansion, non-adherent clusters were subjected to differentiation conditions to generate insulin+, glucose responsive islet-like clusters (see, Example 2). Specifically, non-adherent cell clusters containing an increased number of Pdx-1+ cells were cultured in the presence of an FGF mitogen and at least one additional growth factor or growth factor agonist. Non-adherent spheres were then plated on a coated substratum in the presence of a high-glucose medium, and finally cultured on a coated substratum in the presence of medium containing a standard level of glucose to generate insulin+, glucose responsive islet-like clusters (see Example 2 for a detailed description of these steps of the differentiation protocol).

### EXAMPLE 8

### Differentiation of Non-Adherent Clusters Previously Expanded in Culture

Cells were expanded in culture for 8-12 days, as described in Example 6. Following expansion, non-adherent clusters are subjected to differentiation conditions to generate insulin+, glucose responsive cells and islet-like clusters largely in accordance with the methods outlined in Example 2. Specially, non-adherent cell clusters containing an increased number of Pdx-1+ cells are cultured in the presence of an FGF mitogen and at least one additional growth factor or growth factor agonist. Non-adherent spheres are then plated on a coated substratum to generate insulin+, glucose responsive islet-like clusters (see Example 2 for a detailed description of these steps of the differentiation protocol).

However, the invention contemplates that, rather than transfer the expanded cells from acidic medium (as may be used during the expansion method) back to a more neutral media containing a varying concentration of glucose, the cells may be differentiated in DMEM/F12 buffered to an acidic pH (for example, pH 5.0-7.2 and more preferably pH 6.9-7.1). This alternative differentiation medium is still supplemented with factors, as detailed in Example 2. The glucose concentration in this differentiation medium can vary broadly between 1 mM-20 mM, and this glucose concentration may either remain the same throughout the differentiation protocol or may vary (i.e., beginning at a higher glucose concentration and progressing to a lower glucose concentration as shown in Example 2).

Accordingly, the present invention contemplates differentiation of expanded cells in either medium containing a constant concentration of glucose ranging from 1 mM-20 mM or in medium containing a variable concentration of glucose. In embodiments where the cells are cultured in medium containing a variable concentration of glucose, the cells are first cultured in medium containing a higher glucose concentration (greater than 10 mM) and then transferred to medium containing a lower glucose concentration (less than 10 mM). As stated above, the sequential addition of factors to this medium should remain the same as previously described.

### EXAMPLE 9

### Expansion of Cells Capable of Generating Insulin+, Glucose Responsive Cells in the Presence of Follistatin and/or Exendin-4

In addition to the factors outlined in detail in Example 6, additional factors have been found to influence the efficiency with which progenitor cells are expanded to increase the number of cells capable of differentiating to insulin+, glucose responsive cells. Specifically, we show that follistatin-based factors (e.g., follistatin, follistatin related gene protein, inhibin, other agents that inhibit activin, etc.) and/or GLP-1 agonists (e.g., exendin-3, exendin-4, GLP-1, GLP-1 analogs, etc.) can be used to further increase the expansion of pdx1+ cells in cultures or spheres of insulin− cells.

The results of these studies are summarized in FIG. 5 which shows that progenitor cell cultures that have been expanded according to the methods of Example 6 (4 days in basal medium; 4 days in acidic expansion medium supplemented with forskolin, dexamethasone, insulin, FGF 18, and heparin) prior to their differentiation produced approximately 62 fold more pdx1+ cells than cells differentiated in the absence of the expansion protocol. This effect on pdx1 expression was further augmented if the follistatin-related factor follistatin or a combination of follistatin and the GLP-1 agonist exendin-4 was added to the above list of factors used to supplement the acidic culture medium. Briefly, cultures expanded in forskolin (a cAMP elevating agent), dexamethasone (a corticosteroid), insulin, FGF18 (a FGF family member), heparin (known to potentiate the activity of FGF family members), and follistatin (a follistatin-related factor) contained approximately 281 fold more pdx1+ cells than cells differentiated in the absence of the expansion protocol. Cultures expanded in forskolin (a cAMP elevating agent), dexamethasone (a corticosteroid), insulin, FGF18 (a FGF family member), heparin (known to potentiate the activity of FGF family members), follistatin (a follistatin-related factor), and exendin-4 (a GLP-1 agonist) contained approximately 300 fold more pdx1+ cells than cells differentiated in the absence of the expansion protocol.

FIG. 6 compares pdx1 expression in cell clusters cultured in expansion medium alone versus cell clusters cultured in expansion medium further supplemented with follistatin. Note the increase in pdx1 expression in cultures containing follistatin. FIG. 7 compares pdx1 expression in cell clusters cultured in expansion medium alone versus cell clusters cultured in expansion medium further supplemented with follistatin and exendin-4. Note the increase in pdx1 expression in cultures containing follistatin and exendin-4.

Without being bound by theory, the basis for the expansion of pdx1+ cells following addition of either follistatin and/or exendin-4 to the acidic expansion medium is not yet known. However, given that each of these proteins is mechanistically related to other protein, the invention contemplates the use of not only follistatin but also other proteins or small molecules that are functionally equivalent to follistatin (follistatin based factors). Exemplary related factors include follistatin-related gene protein and inhibin. Additionally, given that much of follistatin's activity is believed to be mediated by its role as an inhibitor of activin (follistatin physically interacts with and inhibits activin protein), the invention contemplates the use of other activin inhibitors (whether they inhibit activin by the same mechanism as follistatin or via a different mechanism) in the expansion protocol. The invention contemplates the addition of follistatin, and/or one of more follistatin-based factors, at any of a number of concentrations. Preferably the final concentration of follistatin or follistatin related factors in the culture medium should be from 1 ng/ml to 1 mg/ml. More preferably, however, the final concentration should be from 100 ng/ml to 400 ng/ml. In the case of the addition of multiple follistatin-based factors, the invention contemplates embodiments in which each factor is added in the above referenced concentration ranges as well as embodiments in which the total concentration of the two or more factors is within the above referenced concentration range.

Exendin-4 is mechanistically related to other proteins, and the invention contemplates the use of not only exendin-4 (in the presence or absence of a follistatin based factor) but also other proteins or small molecules that are functionally equivalent to exendin-4 (GLP-1 agonists). Exemplary GLP-1 agonists include exendin-3, exendin-4, GLP-1 and GLP-1 analogs. The invention contemplates the use of one or more GLP-1 agonists in the expansion medium in the presence or absence of one or more follistatin-based factors. The invention contemplates the addition of exendin-4, and/or one of more GLP-1 agonists (in the presence or absence of one or more follistatin based factors), at any of a number of concentrations. Preferably the final concentration of exendin-4 or other GLP-1 agonists in the culture medium should be from 1 ng/ml to 1 mg/ml. More preferably, however, the final concentration should be from 50 ng/ml to 400 ng/ml. In the case of the addition of multiple GLP-1 agonists, the invention contemplates embodiments in which each factor is added in the above referenced concentration ranges as well as embodiments in which the total concentration of the two or more factors is within the above referenced concentration range.

Additional References

Kaczorowski et al. (2002) Diabetes Metab Res Rev 18: 442-450.

Lumelsky et al. (2001) Science 292: 1389-1394.

Peck et al. (2002) Ann NY Acad Sci. 958: 59-68.

Abraham et al. (2002) Endocrinology 143: 3152-3161.

Petrovsky et al. (2002) Drugs 62: 2617-2635.

Finley et al. (1996) Journal of Neuroscience 16: 1056-1065.

Dell et al. (1997) FEBS Letter 419: 161-165.

Wankell et al. (2001) Journal of Endcrinology 171: 385-395.

Gaddy-Kurten et al. (2002) Endocrinology 143: 74-83.

Maldonado et al. (2000) Journal of Gastrointestinal Sure 4 : 269-275.

de Kretser et al. (2002) Human Reprod Update 8: 529-541.

Welt et al. (2002) Exp Biol Med 227: 724-752.

Schneyer et al. (2001) Mol Cell Endocrinology 180: 33-38.

Edlund (2001) Diabetes 50 Suppl 1: S5-9.

Phillips and de Kretser (1998) Front Neuroendocrinology 19: 287-322.

Mather et al. (1997) Proc Soc Exp Biol Med 215: 209-222.

Thum et al. (2002) Exp Clin Endocrinol Diabetes 110: 113-118.

Aziz and Anderson (2002) Journal of Nutrition 132: 990-995.

Tourrel et al. (2002) Diabetes 51: 1443-1452.

Tourrel et al. (2001) Diabetes 50: 1562-1570.

Egan et al. (2002) Journal of Clin Endocrinol Metab 87: 1282-1290.

Peters et al. (2001) Journal of Nutrition 131: 2164-2170.

Doyle and Egan (2001) Recent Prog Horm Res 56: 377-399.

Bonner-Weir et al. (1993) Diabetes 42: 1715-1720.

Fernandes et al. (1997) Endocrinology 138: 1750-1762.

Githens, S. (1988) J. Ped. Gastroenterol. and Nutr. 7: 486-506.

Lampeter et al. (1995) Exp. Clin. Endocrinol. Diabetes 103 (suppl 2): 74-78.

Offield et al. (1996) Development 122: 983-995.

Ahlgren et al. (1996) Development 122: 1409-1416.

Madsen et al. (1996) Eur. J. Biochem. 242: 435-445.

Edlund, H. (1998) Diabetes 47: 1817-1823.

Apelqvist et al. (1997) Curr. Biol. 7: 801-804.

Githens and Whelan. (1983) J. Tissue Cult. Methods 8: 97-102.

Van Nest et al. (1983) Dev. Biol. 98: 295-303.

Lambillote et al. (1997) J. Clin. Invest. 99: 414-423.

WO95/18856

WO96/17924

U.S. Pat. No. 6,326,201

PCT/US00/03419

PCT/US01/24897

All publications, patents and patent applications are herein incorporated by reference in their entirety to the same extent as if each individual publication, patent or patent application was specifically and individually indicated to be incorporated by reference in its entirety.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method for culturing substantially purified, insulin− cells, wherein the insulin− cells differentiate to insulin+ cells, which insulin+ cells are responsive to glucose.

2. The method of claim 1, wherein the insulin+ cells are pdx1+.

3. The method of claim 1, wherein the insulin− cells are isolated from pancreas.

4. The method of claim 1, wherein the insulin− cells are isolated from duct or tubule tissue.

5. The method of claim 4, wherein the duct or tubule tissue is selected from any of pancreatic duct, hepatic duct, kidney duct, kidney tubule (e.g., proximal tubule, distal tubule), bile duct, tear duct, lactiferous duct, ejaculatory duct, seminiferous tubule, efferent duct, cystic duct, lymphatic duct, or thoracic duct.

6. The method of claim 1, wherein the insulin− cells differentiate to form islet-like structures containing insulin+ cells.

7. The method of claim 6, wherein the insulin+ cells are glucose responsive.

8. The method of claim 6, wherein the islet-like structures contain glucagon+ cells and somatostatin+ cells.

9. The method of claim 8, wherein the glucagon+ cells and the somatostatin+ cells are localized to the periphery of the islet-like structure.

10. The method of claim 1, wherein the insulin− cells are stem cells.

11. The method of claim 10, wherein the stem cells are selected from any of embryonic stem cells, fetal stem cells, or adult stem cells.

12. The method of claim 11, wherein the adult stem cells are selected from any of neural stem cells, neural crest stem cells, pancreatic stem cells, skin-derived stem cells, cardiac stem cells, liver stem cells, endothelial stem cells, hematopoietic stem cells, and mesenchymal stem cells.

13. The method of claim 11, wherein the adult stem cells are derived from an adult tissue.

14. The method of claim 13, wherein the adult tissue is selected from any of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, or olfactory epithelium.

15. A method for differentiating substantially purified, insulin− cells to insulin+, glucose responsive cells, comprising
(a) culturing purified cells as non-adherent spheres;
(b) selecting cells by culturing in the presence of a gp130 agonist;
(c) dissociating the spheres and culturing in the presence of mitogens, wherein at least one mitogen is an FGF family member;
(d) culturing the spheres in the presence of at least two growth factors, or growth factor agonists, wherein at least one growth factor is an FGF family member;
(e) plating the spheres on a coated substratum in high-glucose media; and
(f) culturing the spheres in media containing standard glucose.

16. The method of claim 15, wherein the gp130 agonist is selected from any of cardiotrophin-1, LIF, oncostatin M, IL-6, IL-11, ciliary neurotrophic factor, or granulocyte colony stimulating factor.

17. The method of claim 15, wherein the FGF family member of step (c) or (d) is selected from any of FGF-5, FGF-7, FGF-8, FGF-10, FGF-16, FGF-17, or FGF-18.

18. The method of claim 15, wherein the FGF family member of step (c) or (d) is selected from any of FGF-8, FGF-17, or FGF-18.

19. The method of claim 15, wherein step (c) includes a hedgehog family member selected from any of sonic hedgehog, desert hedgehog, or Indian hedgehog.

20. The method of claim 15, wherein step (c) includes an agonist of hedgehog signaling.

21. The method of any of claims claim 17-20, wherein step (c) includes heparin.

22. The method of claim 15, wherein the growth factors of (d) are family members selected from any of EGF, FGF, IGF-1, IGF-11, TGF-α, TGF-β, PDGF, VEGF, or hedgehog.

23. The method of claim 15, wherein the coated substratum of (e) comprises at least one of poly-L-ornithine, laminin, fibronectin, or superfibronectin.

24. The method of claim 23, wherein the coated substratum is superfibronectin.

25. The method of claim 15, wherein the coated substratum of (e) comprises Matrigel or a cellular feeder layer.

26. The method of claim 15, wherein the high-glucose media of (e) comprises at least 10 mM glucose.

27. The method of claim 26, wherein the high-glucose media comprises at least 11 mM glucose.

28. The method of claim 15, wherein (e) includes at least one factor selected from any of serum, PYY, HGF, or forskolin.

29. The method of claim 15, wherein (e) includes at least one cAMP elevating agent.

30. The method of claim 29, wherein at least one cAMP elevating agent is forskolin.

31. The method of claim 29, wherein the cAMP elevating agent is selected from any of CPT-cAMP, forskolin, Na-Butyrate, isobutyl methylxanthine, cholera toxin, 8-bromo-cAMP, dibutyryl-cAMP, dioctanoyl-cAMP, pertussis toxin, prostaglandins, coiforsin, β-adrenergic receptor agonists, or cAMP analogs.

32. The method of claim 29, wherein at least one cAMP elevating agent is an inhibitor of cAMP phosphodiesterase.

33. The method of claim 15, wherein the standard glucose media of (f) comprises less than 7.5 mM glucose.

34. The method of claim 33, wherein the standard glucose media comprises less than 6 mM glucose.

35. The method of claim 34, wherein the standard glucose media comprises less than 5.5 mM glucose.

36. The method of claim 15, wherein the media of (f) additionally comprises at least one factor selected from any of serum, leptin, nicotinamide, malonyl CoA or exendin-4.

37. The method of claim 15, wherein the insulin− cells differentiate to form islet-like structures containing insulin+ cells.

38. The method of claim 37, wherein the islet-like structures also contain glucagon+ cells and somatostatin+ cells.

39. The method of claim 38, wherein the glucagon+ cells and the somatostatin+ cells are localized to the periphery of the islet-like structure.

40. The method of claim 15, wherein the method for differentiating substantially purified, insulin− cells to insulin+ cells includes expanding the pdx1+ cells within the non-adherent spheres.

41. A method for differentiating substantially purified, insulin− cells to insulin+, glucose responsive cells, comprising
(a) culturing purified cells as non-adherent spheres;
(b) selecting cells by culturing in serum-free media supplemented with cardiotrophin-1;
(c) dissociating the spheres and culturing in serum-free media supplemented with FGF-18 and a hedgehog polypeptide;
(d) culturing the spheres in the presence of at least two growth factors, or growth factor agonists, wherein at least one growth factor is FGF-18;
(e) plating the spheres on a coated substratum in high-glucose media; and
(f) culturing the spheres in media containing standard glucose supplemented with nicotinamide.

42. The method of claim 41, wherein the media of (c) includes heparin.

43. The method of claim 41, wherein the growth factors of (d) are members of a growth factor family selected from any of EGF, FGF, TGF-α. TGF-β, IGF-I, IGF-II, PDGF, VEGF, or hedgehog.

44. The method of claim 41, wherein the media of (d) includes heparin.

45. The method of claim 41, wherein the coated substratum of (e) comprises at least one of poly-L-ornithine, laminin, fibronectin, or superfibronectin.

46. The method of claim 45, wherein the coated substratum of (e) comprises superfibronectin.

47. The method of claim 41, wherein the coated substratum of (e) comprises Matrigel or a cellular feeder layer.

48. A composition comprising an islet-like structure differentiated from substantially purified insulin− cells or progeny thereof.

49. A composition comprising insulin+, glucose responsive cells differentiated from substantially purified insulin− cells or progeny thereof.

50. A composition comprising an islet-like structure differentiated from substantially purified insulin− cells or progeny thereof and a pharmaceutically acceptable carrier or excipient.

51. A composition comprising insulin+, glucose responsive cells differentiated from substantially purified insulin− cells or progeny thereof and a pharmaceutically acceptable carrier or excipient.

52. A method for treating a patient with a condition characterized by impaired responsiveness to glucose, comprising administering to the patient an amount of the islet-like structures of claim 48 or 50 effective to improve glucose-responsiveness.

53. A method for treating a patient with a condition characterized by impaired responsiveness to glucose, comprising administering to the patient an amount of the insulin+, glucose responsive cells of claim 49 or 51 effective to improve glucose-responsiveness.

54. A method of increasing the number of Pdx1− cells in a non-adherent sphere of insulin− cells, wherein said Pdx1− can differentiate to Pdx1+ cells comprising
(a) culturing said insulin− cells to form non-adherent sphere; and
(b) culturing said non-adherent sphere in media comprising an FGF mitogen and a cAMP elevating agent for at least one day, whereby following at least one day in culture in media comprising an FGF mitogen and a cAMP elevating agent the number of Pdx1− cells in said non-adherent sphere which can differentiate to Pdx1+ cells increases.

55. The method of claim 54, wherein said media is acidic media of pH 5.0-7.2.

56. The method of claim 55, wherein said media is acidic media of pH 6.9-7.1.

57. The method of claim 54, wherein said non-adherent sphere of cells is cultured in acidic media prior to addition of media comprising an FGF mitogen and a cAMP elevating agent.

58. The method of claim 57, wherein said media comprising an FGF mitogen and a cAMP elevating agent is acidic media.

59. The method of claim 57, wherein said media comprising an FGF mitogen and a cAMP elevating agent is neutral media.

60. The method of claim 54, wherein said method comprises culturing said non-adherent spheres in media comprising an FGF mitogen, a cAMP elevating agent, insulin and/or a corticosteroid.

61. The method of claim 60, wherein said FGF mitogen is selected from any of FGF-5, FGF-7, FGF-8, FGF-10, FGF-16, FGF-17, or FGF-18.

62. The method of claim 60, wherein said cAMP elevating agent is selected from any of CPT-cAMP, forskolin, Na-Butyrate, isobutyl methylxanthine, cholera toxin, 8-bromo-cAMP, dibutyryl-cAMP, dioctanoyl-cAMP, pertussis toxin, prostaglandins, colforsin, β-adrenergic receptor agonists, or cAMP analogs.

63. The method of claim 60, wherein said corticosteroid is selected from any of dexamethasone, hydrocortisone, cortisone, prednisolone, methylprednisolone, triamcinolone, or betamethasone

64. The method of claim 54, wherein said method comprises culturing said non-adherent spheres in media comprising one or more follistatin-based factors or one or more GLP-1 agonists.

65. The method of claim 54, wherein said method comprises culturing said non-adherent spheres in media comprising one or more follistatin-based factors and one or more GLP-1 agonists

66. The method of claim 64 or 65, wherein said follistatin-based factor is selected from any of a follistatin, a follistatin-related gene protein, or an inhibin.

67. The method of claim 64 or 65, wherein said GLP-1 agonist is selected from any of exendin-4, exendin-3, GLP-1, or a GLP-1 analog.

68. The method of any of claims 54, 60 or 65, further comprising differentiating said non-adherent spheres comprising Pdx-1+ cells to produce insulin+, glucose responsive cells.

69. A method of dissociating a cluster of cells, comprising culturing the cluster of cells in the presence of Protease XXIII.

70. The method of claim 69, wherein said cells are stem cells.

71. The method of claim 70, wherein said stem cells are selected from any of embryonic stem cells, fetal stem cells, or adult stem cells.

72. The method of claim 71, wherein said adult stem cells are selected from any of neural stem cells, neural crest stem cells, pancreatic stem cells, skin-derived stem cells, cardiac stem cells, liver stem cells, endothelial stem cells, hematopoietic stem cells, or mesenchymal stem cells.

73. The method of claim 71, wherein said adult stem cells are isolated from an adult tissue.

74. The method of claim 73, wherein said adult tissue is selected from any of brain, spinal cord, epidermis, dermis, pancreas, liver, stomach, small intestine, large intestine, rectum, kidney, bladder, esophagus, lung, cardiac muscle, skeletal muscle, endothelium, blood, vasculature, cartilage, bone, bone marrow, uterus, tongue, or olfactory epithelium.

75. A composition comprising substantially purified insulin+, glucose responsive cells differentiated by the method of any of claims 1, 15, 41 or 68.

76. An isolated insulin+, glucose responsive cell differentiated by the method of any of claims 1, 15, 41 or 68.

77. Use of insulin+, glucose responsive cells in the manufacture of a medicament to treat a condition in a patient, wherein said condition is characterized by an inhibition of glucose responsiveness.

78. Use of islet-like structures containing insulin+, glucose responsive cells in the manufacture of a medicament to treat a condition in a patient, wherein said condition is characterized by an inhibition of glucose responsiveness.

79. The use of claim 77 or 78, wherein said condition comprises diabetes.

80. The use of claim 77 or 78, wherein said condition comprises an injury or disease of the pancreas.

81. The use of claim 77 or 78, wherein said condition comprises an injury or disease of β-cells.
